(19) United States
(12) Patent Application Publication
(10) Pub. No.: US 2022/0206025 A1
(43) Pub. Date: Jun. 30, 2022

(54) METHOD AND APPARATUS FOR SUBSTRATE HANDLING AND PRINTING

(71) Applicant: ARRAYJET LIMITED, Midlothian, Lothian (GB)

(72) Inventors: Iain Stuart MCWILLIAM, Midlothian, Lothian (GB); Marisa CHONGKWAN, Midlothian, Lothian (GB)

(21) Appl. No.: 17/602,082

(22) PCT Filed: Apr. 14, 2020
(86) PCT No.: PCT/GB2020/050945

§ 371 Date: Oct. 7, 2021

(30) Foreign Application Priority Data
Apr. 11, 2019 (GB) 1905181.2

(51) Int. Cl. G01N 35/10; G01N 33/543

(52) U.S. Cl. CPC G01N 35/10; G01N 2035/00158; G01N 33/543

(57) ABSTRACT

The present invention relates to a method and device for manufacturing microarrays, wherein a microarray comprises a plurality of spots, for testing the interaction of biomolecules. Disclosed herein is a method for enhancing efficiency of overlay printing of spot positions on multiple slides or plates arranged in an array wherein a slide or plate order is provided by rows and columns.

### FIELD OF THE INVENTION

The present invention relates to a method and device for manufacturing microarrays, wherein a microarray comprises a plurality of spots, for testing the interaction of biomolecules.

### BACKGROUND OF THE INVENTION

Microarrays are important in the study of biomolecules such as genomic DNA, cDNA, oligonucleotide sequences, protein, antibodies and the like. Microarrays can be useful in analysis of biomolecular interactions, for example to measure protein binding. Printing of the biomolecules onto a substrate allows analysis to be undertaken on a large number of samples.

Microarrays can be printed on a substrate, suitably a slide, to provide an ordered array of reagents or biomolecules on the substrate. Printing can be by means of an array printer comprising a dispensing printhead such as an inkjet printhead.

Using an inkjet printhead, spots of liquids comprising reagents and/or biomolecules can be accurately located on a substrate. Typically, the dispensing printhead is loaded with reagent or biomolecule. Normally a substrate to be printed is loaded onto a tray and the printhead is moved with respect to the tray in subsequent print passes to print all of the substrates in a complete print job. Multiple trays may be provided in rows and columns each tray comprising substrates onto which reagent is to be printed.

When manufacturing a microarray, it is normally required to print one, two, three or more spots of each liquid onto each of a large number (tens to hundreds) of substrates/slides. In view of this, typically, there will be a very large number (hundreds to tens of thousands) of different liquids to be printed onto the substrates, so the printing process may be lengthy.

Patent application WO 02/11889 discloses a method whereby an inkjet printhead having multiple chambers, each associated with a nozzle, can be used to print multiple different liquids at the same time. The printing can be carried out without cross-contamination between the liquids, despite the fact that the chambers are connected by one or more manifolds internal to the printhead. The liquids are introduced via contiguous groups of nozzles into the associated chambers and printed before they have time to mix by diffusion. Handling multiple liquids therefore offers the possibility of reducing the time taken to do the considerable amount of printing required in the production of microarrays.

If a large number of substrates, for example slides are to be printed, their area may be too large to allow them to be arranged in a plane for easy access during printing. It is therefore only practical to arrange smaller groups of the substrates (slides or plates) in a plane for printing. Slides of the group which are not being printed can then be stored or kept in a different plane from the printing plane. Since the slides do not occupy much volume, it may be convenient to store the slides in a multilayer stack. However, the transfer into and out of storage inevitably occupies some time, conflicting with the need to minimise manufacturing time.

Assays to identify compounds or molecules of interest that may be involved in disease processes or in treating diseases or conditions are available. For example, reverse phase protein arrays (RPPA) of liquid biopsies or cell and tissue lysates are currently used which allow biomarker screening of samples prepared from human sera, saliva, urine, microdissections, or other biologicals fluids or tissues. In such reverse phase protein arrays, protein samples can be printed onto substrate to form microarrays. These microarrays can provide the protein samples at high density. Typically, after the protein samples are printed onto a substrate they are blocked to reduce non-specific binding to the samples and then the substrate is sequentially incubated with an antibody targeted against a biomarker and a complementary labelled antibody conjugate with wash steps between and after each incubation (further steps may be applied to amplify the signal obtained). A quantitative measure of the labelled samples is then obtained to provide details of the level of specific protein present in each printed protein sample. Advances using planar waveguide-based field fluorescence excitation have significantly increased the read out sensitivity (Ayoglu et al, 2011, Expert Reviews, Systematic antibody and antigen based proteomic profiling with microarrays). However, there is an intrinsic read out sensitivity associated with such reverse-phase array applications due to the very small volumes of samples that are deposited on the array surface. As a result, reverse phase protein arrays are typically only suitable for analysis of medium to highly abundant protein targets.

To provide sufficient throughput times, previous assay methods to identify compounds or molecules of interest involved in disease processes or in treating diseases or conditions commonly involve blanket treating the array surface with reagents including wash steps, binding partners, and antigen binding partners. In such circumstances the surface between two spots of an array is similarly treated with reagent(s). This can cause issues with high background noise, particularly if the antigen binding member lacks high specificity for the antigen.

### SUMMARY OF THE INVENTION

Rather than blanket treating an array surface, the present inventors have developed a system for spot on spot printing. In such spot on spot printing systems a high level of accuracy is required when printing a first treatment on a substrate, moving the substrate out of the print bed (to perform interim processes on the printed substrate or to make space for interim printing on other substrates) then printing a second treatment on the substrate. There is a risk that loss of accuracy or inadequate accuracy may introduce printing errors, for example due to minor variations in print-bed height and substrate position which would be sufficient to invalidate assays due to the high tolerances required in microarray printing. The present invention minimises the risk of such inaccuracy.

The methods of the present invention can be particularly useful for combinatorial library screening. Combinatorial library screening can be an assay where a library of first potential binding partners are screened for their interaction with one or more second potential binding partners. Maximising throughput is important during combinatorial library screening as increasing the number of potential binding partners exponentially increases the number of combinations of first and second potential binding partners to be screened. This is particularly the case for combinatorial library screening assays where a plurality of second potential binding partners are to be screened. Organisational efficiency is required to optimise combinatorial library screening methods.

Due to the limitations with the reverse phase protein arrays method when analysing interactions between binding molecules, it would be advantageous if a first spot could be printed comprising a first member of a potential binding partner pair and then a second spot was printed overlaying the first spot wherein the second spot comprises a second member of a potential binding partner pair. Overlay printing (also known as spot on spot printing) is considered by the present inventors to reduce the overall background signal when trying to detect interactions between the first member of a potential binding partner pair and the second member of the potential binding partner pair. However, as the repeatability of the position and orientation of the substrate in spot-on-spot printing is critical, particularly in applications where at least one first spot is printed on a substrate, and the substrate is moved out of the print bed (to perform interim processes on the printed substrate or to make space for interim printing on other substrates) then at least one second spot is printed on the substrate, spot on spot printing faces technical challenges in printing on large numbers of slides. If, during the printing of at least one second spot the substrate (slide) is not in the same position and orientation as was used in the printing of the previous layer, due to the tolerances required by microarray spot-on-spot printing it is highly likely that the next spot will not significantly overlay the previous spot, invalidating the assay. For example, minor variances in the mounting rails of a print bed for a substrate held in a first position for the first printing and a second position in a second printing may prevent accurate spot-on-spot printing, and invalidate the assay.

The present inventors have determined a method that uses spot on spot printing and provides for improved detection of the analyte. Without wishing to be bound by theory, it is considered the improved detection is provided due to
the reduced background observed, for example when compared with reverse-phase protein microarray and
improved reaction kinetics, as the proximity of the specific binding member, for example an antibody or fragment thereof and the analyte/antigen (provided for example by the cell lysate) is improved as printing of both components provides these to the same location.

Accordingly, a first aspect of the invention, provides a method for enhancing efficiency of overlay printing of spot positions on multiple slides or plates arranged in an array wherein a slide or plate order is provided by rows and columns, the method comprising the steps:
printing at least one spot of a first test material comprising a first type of first potential binding partner pair onto the first row (r1) of plates in an array of n columns in a printing order to provide the first test material on at least plate r1n1 and a replicate plate r1n2
printing at least one spot of a second test material comprising a second type of first potential binding partner pair onto a second row (r2) of plates in an array of n columns in a printing order to provide a second test material on at least plate r2n1 and a replicate plate r2n2,
printing spots of a first overlay material comprising a first type of second potential binding partner pair to overlay the spots of the at least first test material and/or the spots of the at least second test material wherein when the overlay material is printed, the plate is provided at the same position at which the test material was applied.

Suitably between the printing of the test material and overlay material the plates in the array are rearranged (reordered) to reorder the plates to maximise printing efficiency of the overlay material.

Suitably following rearrangement or reordering of the plates the first overlay material is provided to overlay the spots of the at least first test material and/or the spots of the at least second test material without requiring movement of the printhead between rows.

Advantageously the method allows higher throughput of printing and thus screening.

Advantageously, each plate or slide needs to be in the same slide position and ‘row’ for Printing 1 (lysate or first binding partner) and Printing 2 (hybridoma or second binding partner). This ensures positional accuracy.

As will be appreciated herein a tray position describes a row position and a slide position describes a column position.

In the present application the slides are identified according to
Slide position (column): S
Tray: T
Sample name (for example Lysate): L

Thus a print run might include

S1T4L16=slide 1, tray 4, lysate 16

S1T3L11=slide 1, tray 3, lysate 11

S1T2L6=slide 1, tray 2, lysate 6

S1T1L1=slide 1, tray 1, lysate 1

Advantageously in embodiments, the method allows the printing of multiple different lysates, for example 100 different lysates in one print run and then for these to be placed in the same same location where they were printed such that an overlay print run can be provided.

Advantageously the method allows determination of positive interactions between a specific binding member (affinity reagents, suitably antibodies) and an analyte, suitably unknown analytes, suitably cells or cell-derived products, on a printed microarray format of unknown analytes. Suitably the analyte may comprise at least one cell or cell lysate, for example the analyte can be a cancer cell or cancer cell lysate or a cell or lysate from the blood or tissue of a test subject. Suitably the analyte may be a protein from at least one cell lysate.

Suitably, to allow detection of binding between binding partners at improved detection levels, improved blocking and labelling techniques may advantageously be utilised to enhance the detection of binding partners, for example binding protein partners in the protein/cell samples printed onto the substrate. Additionally, the substrates onto which the protein/cell samples have been printed may be optimised to allow for improved binding and presentation of the protein samples.

Difficulties in undertaking overlay printing include the very large number of different liquids to be printed onto the substrates, the potential for the printing process to be very lengthy, and the accuracy requirement of the printing technology to be able to print one spot on top of another.

The loading of the dispensing printhead can require a significant amount of reagent or biomolecule relative to the amount required for a print run and thus it is advantageous to undertake printing such that the number of loading steps is minimised. Further, the cleaning of the printhead before the introduction of the next set of liquids takes time, thus it is advantageous to undertake printing such that the number of cleaning steps is minimised; therefore, once a set of liquids has been introduced into the printhead, advantageously they should be printed onto all the slides before another set of liquids is loaded.

To maximise the increase in throughput achieved by minimising the number of cleaning steps, use of a large number of slides is beneficial. Printing onto all the slides before another set of liquids is loaded limits the assay by the number of slides used. As previously stated, where the surface area of the slides is too large to allow them to be arranged in a plane for easy access during multi-layer printing, it is practical to arrange smaller groups of the substrates (slides or plates) for printing, storing them in a multilayer stack between layers of printing.

To overcome the issue of a binding partner becoming contaminated or mis-printing midway through the printing of a combinatorial screening assay, as well as preventing misprinting due to printing the plates in different positions and orientations between layers of printing, the inventors have developed a method of selective substrate (slide or plate) reordering, this maximises throughput by minimising cleaning steps without being limited to the available printing area.

Suitably in the method, multiple spots of a test material are provided to replicate slides or plates in a row (r) or particular tray (t as described above), for example all plates of r1n1 and r1n2 are printed sequentially with the at least one spot of the first test material.

Suitably the overlay material is provided to the spots of the test material when the plates are provided at the same position in the printer as when the test material was applied, along a continuous row of slides. For example, the printing of the overlay material does not require the printhead to move between rows when overlaying the first material.

Suitably in the method the slides may be rectangular with each slide having two long edges and two short edges.

Suitably in the method, in each row, a subgroup of a number of slides (Sr)′ may be printed with a first test material (t1). This provides replicate slides with each replicate slide providing a first position of a column in a row (for example: r1n(Sr)′t1, r1n(Sr)′t1, r1n(Sr)′t1, r1n(Sr)′t1, r1n(Sr)′t1).

Advantageously, to process 10,000 hybridomas, 5 slides per test material (lysate) may be provided to allow 5 rounds of 20 concatenated print runs.

Advantageously subgroups of 1, 2, 3, 4, 5, 6, 8, 12, 24, 25 slides per test material (lysate) may be provided. In embodiments of the method, trays of 25 slides may be provided. In such examples a tray is provided in a row, but it will be understood that the tray provides a subgroup of the row. For 2, 3, 4, 6, 8, 12 and 24 the maximum number of slides per tray can be 24. However, as will be appreciated if trays are provided which incorporate more than 25 slides, different formats of printing may be provided.

In embodiments, for example in a case where the subgroup is 5 slides, in the method, in each row, five replicate slides are printed with a first test material (t1) with each replicate plate providing a first position of a column in a row (for example: r1n1t1, r1n2t1, r1n3t1, r1n4t1, r1n5t1).

Suitably, the test material (for example Lysate number (L)) to use can be determined based on the following input (ArrayPlex determination):
Binding partner Print Run (for example Hybridoma Print Run Number (H))
Slide Number (S)
Tray Number (T)
Slides per subrun (Sr)=5
Runs/cycle (Rc)=20
Slides per total run (St)=100
Slides per tray=25
Based on the data above, the following offsets are calculated:

Binding partner Offset (e.g. HybridomaOffset)=MOD(Rc*(Sr−(H−1)), St);
Which provides (based on above):

Binding Partner Offset (e.g. HybridomaOffset)=Ho=MOD(20*(5−(H−1)), 100);

SlidePosition=Sp=MOD(S−1, 25)+1;

SlideOffset=So=MOD((SlidePosition−1)*20, 100);

SubrunOffset=Sro=QUOTIENT(Sp−1,5);

TrayOffset=To=(T−1)*5;

Test Material (e.g. Lysate)=L=MOD(Ho+So+Sro+To, 100);

Where MOD is the module operation (remainder after division of one number by another). FIG. 19 shows the results of use of these parameters. As will be appreciated, if the number of slides in the subrun is changed from 5 then the ArrayPlex determination is altered accordingly.

Suitably, in such embodiments at least 5 replicate slides are arranged in a row with adjacent slides being arranged in a row such that the short edges of the slides are adjacent to each other in a row. Suitably the slides are arranged in a column such that the long edges of the slides are adjacent to each other in a column.

An embodiment of this selective substrate reordering is illustrated in FIGS. 13-15.

Suitably as illustrated in FIG. 13, 5 replicates of at least a first test material are printed in row 1, columns 1, 2, 3, 4, and 5. Suitably a second test material may be printed at row 1 in columns 6, 7, 8, 9 and 10. Suitably a third test material may be printed at row 1 in columns 11, 12, 13, 14 and 15. Suitably further test materials may be printed at row 1 in further columns for example as illustrated to row 1, column 25.

Suitably as illustrated a further test material may be printed at row 2 column 1, 2, 3, 4 and 5. Suitably in the second row additional test material may be printed at row 2 column 6, 7, 8, 9 and 10 etc.

Suitably as illustrated a further test material may be printed at row 3 column 1, 2, 3, 4 and 5. Suitably in the third row additional test material may be printed at row 3 column 6, 7, 8, 9 and 10 etc.

Suitably as illustrated a further test material may be printed at row 4 columns 1, 2, 3, 4 and 5. Suitably in the fourth row additional test material may be printed at row 4 columns 6, 7, 8, 9 and 10 etc.

Thus in this embodiment, blocks of 5 slides or plates in each row may be provided with an individual test material. For illustrative purposes, FIG. 13 has been subdivided into 5 subgroups, however this table is intended to be continuous with 25 columns by 5 rows. As will be appreciated, a first material may be printed or deposited not only to four rows but could be extended as necessary to provide for the total number of binding partners to assay. This is advantageous as the batch sequence printing allows the use of slides printed in for example a second/third/fourth or fifth batch to be used in a second print run to print the second material spot on spot.

This results in a distinct ordering of plates/slides comprising at least one test material per plate/slide, wherein each plate/slide can be defined by a row (tray) and slide (column) position. This is illustrated for example in FIG. 14.

Suitably, prior to printing of an overlay material, the slides/plates are selectively reordered so that a substrate retains the same slide and tray location, but is reordered to allow first, second and third batches to be printed with a different overlay material in the same print run, multiplying the number of samples being tested and thus increasing the throughput.

This selective reordering is such that an overlay material may be printed consecutively across a single row, as shown in FIG. 15, by the printing of overlay 1 on row 1, columns 1-25, Overlay 2 on row 2, columns 1-25, etc. This minimises changing the material in the printhead and thus increases throughput by reducing the number of cleaning steps.

Concurrently, by printing the overlay material with the slide in the same column and row position as it was during the printing of the test material, the variances in printbed height and substrate position are minimised, ensuring positional accuracy and permitting accurate, repeatable spot-on-spot printing where the substrates must be moved between layers of printing (e.g. stored in multi-layer stacks) due to being collectively larger than the available printing area.

An alternative embodiment of overlay printing is to print the same overlay material on multiple rows. This produces a “repeat” of each substrate in a tray. This embodiment protects against contamination or misprints midway through the print run—for instance in strategies where the tray length is 5 (therefore the unit cell is 5 columns by 5 rows) and the printing of the overlay material misprints during row 4, all test materials will have at least 3 repeats, which is sufficient for statistical significance (although 5+ is preferred).

Suitably repeats of the same test could also be provided by test material repeat slides and then use of the same overlay material.

An alternative to the above embodiment is where the slides are not reordered between printing layers; in such a scenario where the overlay material misprints during row 4, there would be no experiments of the test material in the fifth row. As these experiments can be considerably time consuming and expensive, such scenarios are particularly wasteful. The present method is particularly suited to overlay materials which are expensive, rare, time consuming or prone to misprinting.

Suitably four rows and 25 columns are provided for each overlay print run, and five print runs of overlay test material are provided, wherein the slides are reordered such that:
1. the slide of the first row position and first column position of the first test material print run is provided as the first row position and first column position of the first overlay material print run.
2. the slide of the first row position and second column position of the first test material print run is provided as the slide of the first row position and second column position of the second overlay material print run,
3. the slide of the first row position and third column position of the first test material print run is provided as the slide of the first row position and third column position of the third overlay material print run,
4. the slide of the first row position and fourth column position of the first test material print run is provided as the slide of the first row position and fourth column position of the fourth overlay material print run;
5. the slide of the first row position and fifth column position of the first material print run is provided as the slide of the first row and fifth column position of the fifth overlay material print run;
6. such that the row and column position of each slide in the lysate print runs is maintained in the print run with the overlay material.

Another embodiment of the invention is illustrated in FIGS. 17-18, using rows (Tray 1-4 repeated 5 times, (T)) by 25 columns (Slide position, S) of substrates to assay 100 test materials (Lysates (L)) with four overlay materials (T).

Initially, the test materials are printed along the rows (i.e. row 1, columns 1-25, then row 2 columns 1-25, then row 3 columns 1-25 etc.) as shown in FIG. 17, then the substrates are selectively rearranged as shown in FIG. 18 and the overlay materials are printed along the rows (i.e. row 1, columns 1-25, then row 2 columns 1-25, then row 3 columns 1-25 etc.).

Slide movement from printing of the first test material to the second overlay material can be described as provided in the attached figures.

Suitably a first potential binding partner (test material) may be an analyte, for example an analyte may be a protein, a protein fragment, an intact cell, a receptor provided on an intact cell, a receptor provided in a cell lysate, a fusion protein, a nucleic acid sequence or the like.

Suitably a second potential binding partner (overlay material) may be a specific binding molecule, suitably selected from a group comprising an antibody or a fragment thereof (for example single chain antibodies), a small molecule, an aptamer, a nucleic acid molecule, for example siRNA, DNA, PCR amplicon or a synthetic biological molecule, for example a chimeric protein.

It will be understood that the position of the first potential binding partner as a component of the test material and the second potential binding partner as a component of the overlay material may be inverted, with the first potential binding partner as a component of the overlay material and the second potential binding partner as a component of the test material.

Suitably there is provided a method for detection of analyte to a specific binding molecule, for example an antigen binding member (antibody or fragment thereof), the method comprising:

i)
a) printing a test material, for example cell or suspension of cells or a cell lysate composition or part thereof onto a substrate;
b) printing an overlay material, for example an antigen binding member with binding specificity to a test material for example with binding specificity to an analyte to be detected onto the cell or suspension of cells or cell lysate composition or portion thereof provided on the substrate in step a);
   and then
c) incubating the test material and overlay material to allow binding between the test material and overlay material to be detected to occur, for example antigen binding member to allow any specific binding between the antigen binding member and the analyte to be detected to occur;
d) detecting binding of the test material and overlay material, for example detecting antigen binding member to analyte following step c).

It is considered an improvement in detection of analyte will be provided by the present method over detection by reverse phase protein array (RPPA) methods. Suitably an improvement of detection at least two fold, at least three fold, at least four fold, at least five fold, at least six fold, at least seven fold, at least eight fold, at least nine fold, at least ten fold of the spot on spot printing over RPPA is detected.

Suitably, an antigen binding member can be provided by an antibody or a hybridoma or another antibody producing cell, or an aptamer or a small molecule or any other affinity reagent.

Suitably, the method comprises printing a substrate with a cell or suspension of cells or a cell lysate composition or part thereof and then exposing the cell, suspension of cells or cell lysate composition or portion thereof to a specific binding molecule/an antigen binding member by printing of the specific binding molecule/antigen binding member directly onto the analyte wherein the specific binding molecule/antigen binding member and cell or suspension of cells or cell lysate or portion thereof are incubated for about 0.1, 1, 5, 10, 20, 30, 40, 50 hours to allow any specific binding between the antigen binding molecule and the analyte to occur.

Suitably, the cell, suspension of cells or a cell lysate composition or part thereof may be or from an animal cell, in particular a human cell, a bacterial cell, a fungal cell, or plant cell. Suitably the cell, suspension of cells or a cell lysate composition or part thereof may be from a cancer cell. Suitably the cells or a cell lysate composition may be from bacteria. Suitably the cells or cell lysate may be from a plant. Suitably the cell lysate composition may be purified peptides or proteins.

Suitably the detecting step may utilise any immunohistochemical method, for example fluorescence, colorimetry, quantum dots, biotin/avidin, or a label free detection technology such as surface plasmon resonance. Identification of a positive association between an analyte and a specific binding molecule/an antigen binding member can be determined by suitable techniques known in the art for example, fluorescence, colourimetric immunoassays, polymetric methods. Suitable fluorescent labels may include, for example, fluoroscene, isothiocyante, didansyl chloride, lanthanides or other fluorescent labels known in the art.

Suitably the method may further comprise a step of comparing the binding pattern of a specific binding molecule to a first cell or suspension of cells or a cell lysate composition or part thereof with the binding pattern of the specific binding molecule to a second cell or suspension of cells or a cell lysate composition or part thereof.

Suitably, the substrate may be any substrate commonly used in biological testing, for example glass slides, functionalised glass slides, plastic, nitrocellulose, nylon membrane, SPR prism, MEMS devices, microfluidic chip, polystyrene, polycarbonate, PVDF (polyvinylidene fluoride), metals or composites and mixtures of these materials, etc. Suitably the substrate may be coated with a composition or compound which aids binding of cells or cell suspensions or cellular material to the substrate. Suitably a composition or compound may be provided to the substrate to assist in the formation of discrete spots or patterns on the surface of the substrate when the analytes are printed onto the substrate. Suitably, the sensitivity of the method can be increased by printing multiple droplets of a cell or suspension of cells or a cell lysate composition or part thereof onto the same location of an absorptive substrate which captures an analyte, for example protein, within the initial pores/binding sites they encounter whilst allowing solvent and ionic solutes to pass. This should allow an increase in the amount of immobilised analyte, for example protein per unit area to be achieved.

After incubation, any association between the analyte printed on the substrate and the antigen binding member printed on the analyte can be detected. The detection between the analyte and antigen binding member may be by any method known in the art. Suitably, detection can be undertaken using a labelled affinity reagent that can associate with the specific binding molecule/antigen binding member. The labelling of the affinity reagent may be by, for example, use of a fluorescent label, a colorimetric label, a radiolabel or enzyme for enhanced chemiluminescence.

The methods of the invention allow identification and characterisation of previously unknown or undefined analytes for example receptors, or proteins on cells or cell derived compositions, for example the compositions may be secreted from cells such as cytokines or be compositions which are released upon lysis or permeabilisation of the cell for example cytoplasmic, nuclear or cell member components.

Cell lysate compositions can include biopolymers such as DNA and RNA as well as proteins, glycoproteins, glycans, lipids and glycolipids or any other biopolymer. The cellular composition may be printed onto a substrate directly or suitably, the substrate may be functionalised to allow binding or improved binding of the cells or cellular composition. Suitably cells or cellular compositions may comprise a single cell or cell suspension from normal or cancerous tissue. Suitably tissue may be selected from heart, brain, liver, prostate, breast, colon, lung, skin, or other cancerous tissue of the body.

In examples the method of the invention provides a high throughput method of identifying hybridomas and their target antigens/analyte wherein the target antigen/analyte is unknown. In such an example there may be created a primary library of hybridomas from B lymphocytes isolated from an animal, in particular a human, with cancer or cancers. Suitably discrete droplets of a secondary library of cells or cell lysates are printed onto a substrate. For example, the cells or cell lysates may be from tissue biopsies from healthy or disease carrying, for example cancer, animal sources. The cells or cell lysates may be printed at predefined locations on the substrate to provide an array of lysate features. A primary library of hybridoma supernatants may be printed precisely on top of the cells or cell lysates printed on the substrate and incubated to allow binding between the antibody provided by the hybridoma supernatants and the target antigen/analyte provided in the printed cell or cell lysate composition.

The method may further comprise the step of washing the substrates to remove unbound antigen binding member which has not specifically bound to the cell or cell lysate composition after the incubating step. Suitably the detecting step may comprise detection of positive/specific antigen binding member to antigen/analyte binding using a labelled secondary antibody, for example a fluorescently labelled antibody.

Suitably the detecting step allows quantification of the binding of the antigen binding member. Suitably the detecting step or a further step allows identification of an antigen/analyte bound by the antigen binding member printed onto the cell or cell lysate composition.

Suitably, prior to the step of printing in step b) there is provided a step of blocking the cells or cell lysates. Suitably a chemical or biological blocking agent may be used. Suitably bovine serum albumin solution may be used.

Suitably an antigen binding member may be provided using a hybridoma. Hybridomas may be provided by known methods for example an animal may be immunised with an antigen or a plurality of antigens. The spleen of the animal may be removed and broken up to form a suspension and the suspended spleen cells may be fused with myeloma cells and cultured for several days such that unfused spleen and myeloma cells die and fused myeloma and spleen cells survive.

Alternatively, antigen binding members may be provided by libraries of hybridomas or antibodies or the like. Alternatively, hybridomas can be produced by synthetic cloning techniques or by using B cells from the spleen of a subject exhibiting a disease state or infection.

Suitably the antigen binding members, for example, an antibody or hybridoma, may be provided onto the cell or suspension of cells or a cell lysate composition or part thereof printed onto the substrate by non-contact piezo inkjet printing such that they are brought into contact with the analyte and exposed to the analyte to allow specific binding between the antigen binding member and the analyte. Alternatively, the antigen binding members can be provided to the substrate by any other deposition or printing technology which enables spot on spot printing. Suitably a sample or biopsy of an individual's tumour may be taken and the sample printed onto a substrate and antigen binding members, antibodies or hybridomas may be screened against these printed samples and specific binding of an antigen binding member to the unknown analyte provided on the substrate can be determined.

The identification of antigen binding members which are capable of specifically binding to analytes from cells or cell suspensions can allow the development of new therapies or treatment of diseases and conditions. For example, the identification of therapeutic antibodies, in particular therapeutic monoclonal antibodies.

The screening methodology enables the high throughput and rapid analysis of large library populations of immortalised B-cells to identify those B-cell clones which are capable of producing antibodies which are able to specifically bind analytes provided on the substrate.

The methodology disclosed herein may be used to identify unknown analytes printed on a substrate that can specifically bind with unknown antigen binding members. Unknown antigen binding members can be provided from a library or the like. Suitably, a cell lysate library may be prepared from biopsy samples or cultured cells. Suitably, the cell lysate library may be obtained by homogenizing cells in a lysis buffer, for example RIPA buffer. Suitably the lysate concentrations may be altered to provide a concentration of 500 μg/μl. The lysate library may be printed onto the substrate, for example nitrocellulose under specific printing conditions, for example 4° C. and 75% RH. The substrate may be incubated at 4° C. and 75% RH overnight to allow immobilisation. Suitably at least a second incubation period may be provided, for example 30° C. for 1 hour to complete the immobilisation. Suitably the substrate may be blocked, for example using 2.5% BSA (IgG free). Blocking may be for around 90 minutes at room temperature. The substrate may be washed with a suitable buffer and dried, for example with centrifugation. The substrate may be placed in an arrayer for further printing thereon. A hybridoma/antibody library may be provided by printing on top of the lysate array. The hybridoma/antibody library may be provided at a particular concentration, for example 0.01 to 10 μg/ml. Suitably the hybridoma/antibody library may be provided in RPMI media and glycerol, for example 80% RPMI and 20% glycerol. The substrate may be incubated overnight, for example at 4° C. and 75% RH. The substrate may then be washed and dried. A secondary antibody may be applied to the substrate or to the printed regions of the substrate. The substrate may be incubated, for example for 90 minutes at room temperature. The substrate may be washed and then dried. Binding of the hybridoma/antibody library to the lysate array may be detected using any means suitable in the art. The data obtained from the detecting step may be analysed.

Suitably the method of the present invention may use only 100 pL of ˜1 mg/ml of lysate and 100 pL of antibody per test.

According to a further aspect of the present invention there is provided a method of diagnosing a disorder, the method comprising

determining the presence of an analyte in a test material sample using a method of the first aspect of the invention,

wherein when a specific binding molecule (provided in an overlay material) and analyte complex is detected it is indicative of the disorder.

As will be appreciated, the detection of the specific binding molecule and analyte complex may be required to be altered from a control level of binding between the specific binding molecule and analyte complex to be indicative of the disorder.

According to a further aspect of the present invention there is provided a system to provide the method of the first aspect of the invention wherein the system comprises a printer adapted to print

a) one of,

i) a cell or suspension of cells or a cell lysate composition or part thereof, or

ii) a specific binding molecule with binding specificity to an analyte onto a substrate;

b) the other of

i) a specific binding molecule with binding specificity to an analyte to be detected onto the cell or suspension of cells or cell lysate composition or portion thereof provided on the substrate in step a) i), or

ii) a cell or suspension of cells or cell lysate composition or portion thereof provided onto a specific binding molecule with binding specificity to an analyte to be detected provided on the substrate in step a) ii).

Suitably the system may comprise an environmentally controlled module around the printer or printhead. Suitably the module provides a temperature controlled environment around the printer and the slide being printed. Suitably the temperature may be controlled within a range of 0 to 25 degrees C., suitably 0 to 10 degrees C., suitably 2 to 4 degrees C. Suitably the module provides for a temperature controlled environment around the printer of 4 degrees C.+/−2 degrees. Suitably an environmentally controlled module may be an insulated chamber to control the temperature at which a slide is printed. Suitably such a chamber may be sealed such that humidity within the chamber can be controlled.

Controlling the environment of printing and storing of slide/substrates may advantageously prevent the drying of the printed materials prior to and during incubation such that binding between first and second binding members may occur. Suitably control of the environment of the slides minimises denaturing of the materials used in printing, including the test material(s) and overlay material(s). Controlling the humidity may regulate the rate of diffusion of solvents (including water) into and out of spots, regulating droplet wetting on the substrate and the time permitted between spot-on-spot printing before a compound therein is at risk of denaturing due to drying out.

Suitably the system comprises an environmentally controlled module around the printer/printhead and such that the printed slides can be incubated within the module.

Suitably an environmentally controlled module may allow for the humidity around the printer/printhead during printing of a slide and/or incubation of a slide to be controlled. Suitably the humidity may be controlled such that it is 60% RH during printing and 80% RH during incubations. Suitably a chamber which may be sealed to allow temperature and/or humidity to be controlled at the slide when printing and storing of the slide between print runs may be provided by a low volume chamber. Suitably a low volume chamber may have an air volume of less than 4 cubic metres, suitably less than or equal to 2 cubic metres. As will be appreciated in the art, a lower air volume is typically easier to control.

According to a further aspect of the present invention there is provided a kit to provide the method of the first aspect of the invention wherein the kit comprises a substrate onto which one of,

i) a cell or suspension of cells or a cell lysate composition or part thereof, or

ii) a specific binding molecule with binding specificity to an analyte

is printed, for use in a printing system which can print the other of
a specific binding molecule with binding specificity to an analyte to be detected onto the cell or suspension of cells or cell lysate composition or portion thereof provided on the substrate in step i), or
a cell or suspension of cells or cell lysate composition or portion thereof provided onto a specific binding molecule with binding specificity to an analyte to be detected provided on the substrate in step ii).

The kit may comprise instructions for using the kit. The kit may comprise reagents for detecting the binding of the specific binding member and the analyte.

### BRIEF DESCRIPTION OF THE FIGURES

There will now be described, by way of example only, various embodiments of the invention with reference to the following drawings, of which:

FIG. 1 illustrates a functionalised glass slide printed with analyte/antigen (e.g. cell lysate) library;

FIG. 2 illustrates an analyte/antigen binding agent (e.g. hybridoma) library printed directly on top of antigen library;

FIG. 3 illustrates non-binding analyte/antigen binding agents after a washing step being washed away;

FIG. 4 illustrates specific binding of analyte/antigen being detected with a labelled secondary affinity reagent (e.g. antibody);

FIG. 5 illustrates a high resolution scan of an analyte/antigen binding agent library printed directly onto a printed lysate library

FIG. 6 illustrates the data generated from a spot on spot printing example in which four different antibodies were screened at four different concentrations against sixteen cell lysates. In parallel, two RPPA experiments were performed comparing the reactivity of two control antibodies (EGFR and 2C8) at a single concentration (0.1 μg/mL) against fourteen cell lysates (A431, A549 and SKMEL28) which were included in the spot on spot printing example. In the spot on spot array image the box labels highlight sets of four spots per antibody+lysate combination and the data on the graphs represent 0.1 μg/mL antibody (on the image this is the third spot from the top of each box);

FIG. 7 illustrates a spot on spot study wherein four antibodies and supernatants at four concentrations were screened against 16 cell lysates;

FIG. 8 illustrates the data generated from a comparison study of the reactivity of two control antibodies (EGFR and 2C8) against three cell lysates (A431, A549 and SKMEL28);

FIG. 9 illustrates a RPPA experiment wherein antibody 2C8 was provided at 0.1 μg/mL vs cell lysates A431, A549 and SKMEL28;

FIG. 10 illustrates a RPPA experiment wherein EGFR antibody is provided at 0.1 μg/mL vs cell lysates A431, A549 and SKMEL28;

FIG. 11 illustrates the data generated from a comparison of the interactions of four lysates (A431, A549, SKMEL28 and HT29) with antibody 2C8 (0.1 μg·ml) and shows a >10-fold higher in signal using spot-on-spot (B) than RPPA (A).

FIG. 12 is a plan view of apparatus for printing.

FIG. 13 A illustrates a print run of test materials on a configuration of substrates in horizontal arrangement of subgroups and figure B with vertical arrangement of subgroups

FIG. 14 illustrates the selective reordering of the substrates of FIG. 13

FIG. 15 illustrates the printing of overlay materials on the reordered substrates illustrated in FIG. 14.

Alternative FIG. 16 illustrates a print run of test materials on a configuration of substrates, the selective reordering of the substrates and two printing strategies for overlay materials

FIG. 17 illustrates tray arrangement of test materials on plates

FIG. 18 illustrates a printing pattern for the first layer of printing of test materials for 20 different test materials

FIG. 19 illustrates a printing pattern for an overlay material.

FIG. 20 illustrates use of module operation using parameters specified herein to illustrate movement of slides during printing.

FIG. 21 illustrates in A that all the slides in position 1 are designated as S1 and in B that slides in the same tray or row are designated by second positional indicator T1, T2, T3 or T4 with lysates in this example in batches of 5.

FIG. 22 illustrates printing of second binding member (hybridoma) and retaining position of first printed binding member (lysate) in reordered slides.

FIG. 23 A, B, C and D illustrates FIG. 13 B in expanded view

FIG. 24 illustrates FIG. 14 in expanded view, and

FIG. 25 illustrates FIG. 15 in expanded view

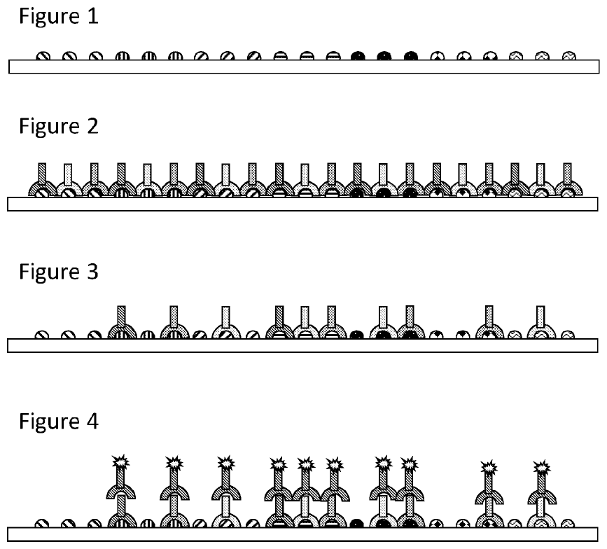

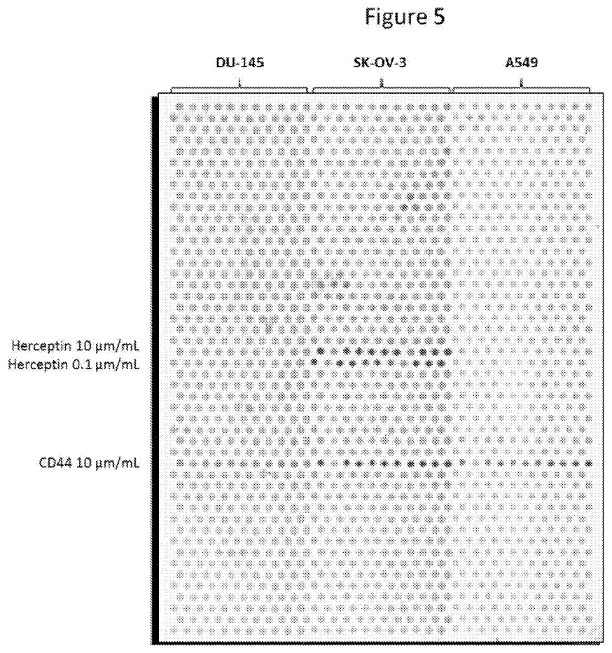

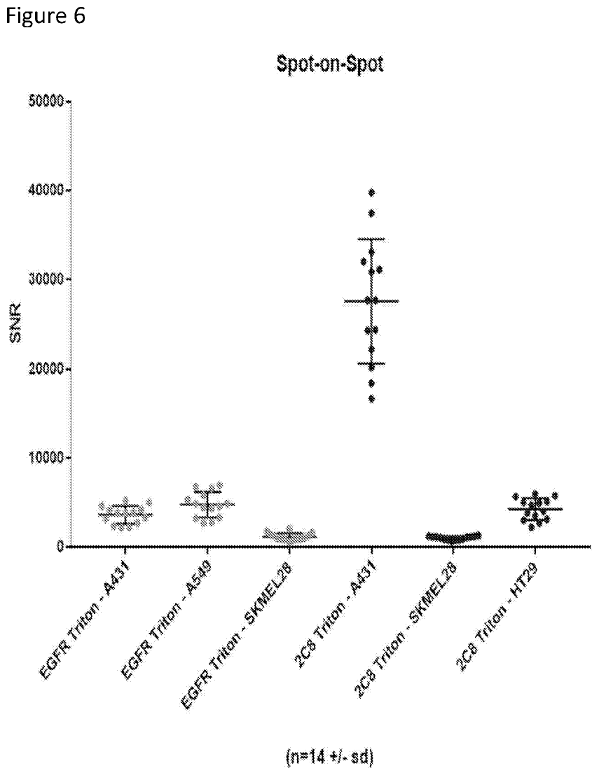

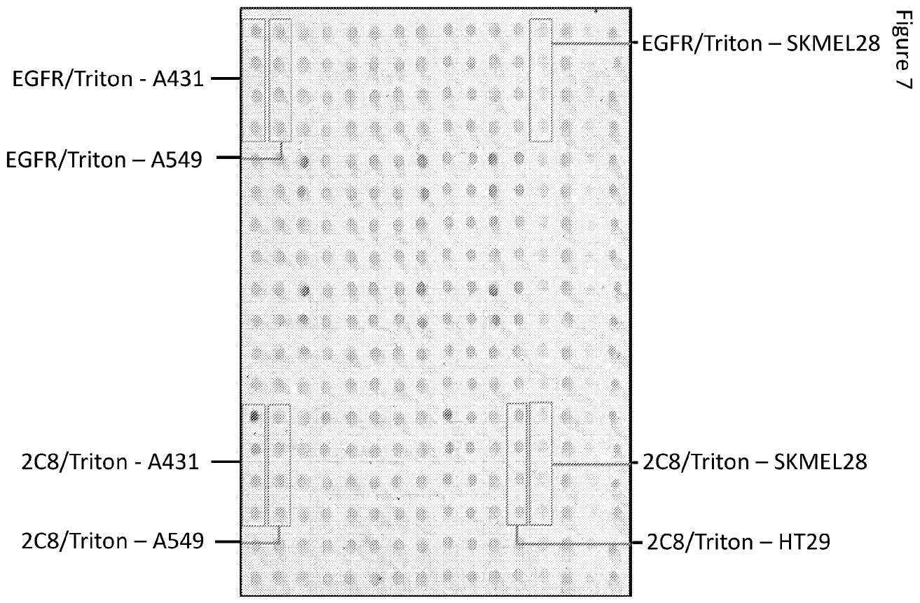

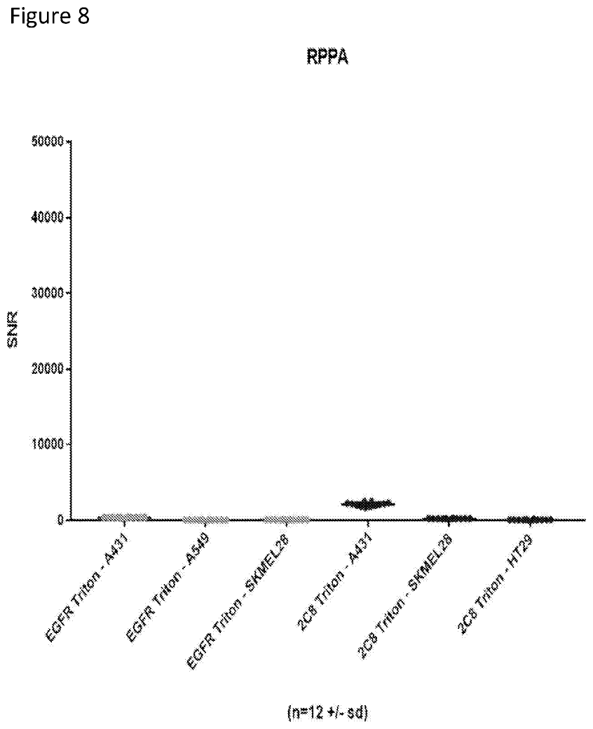

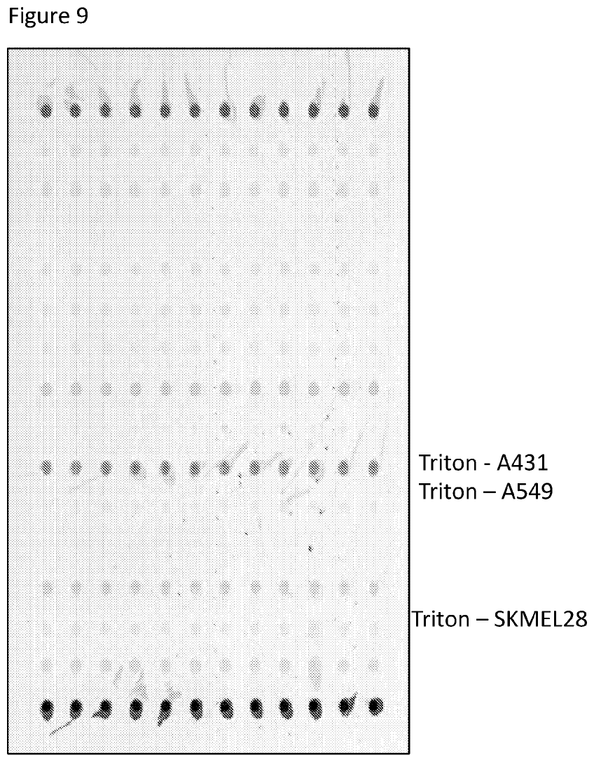

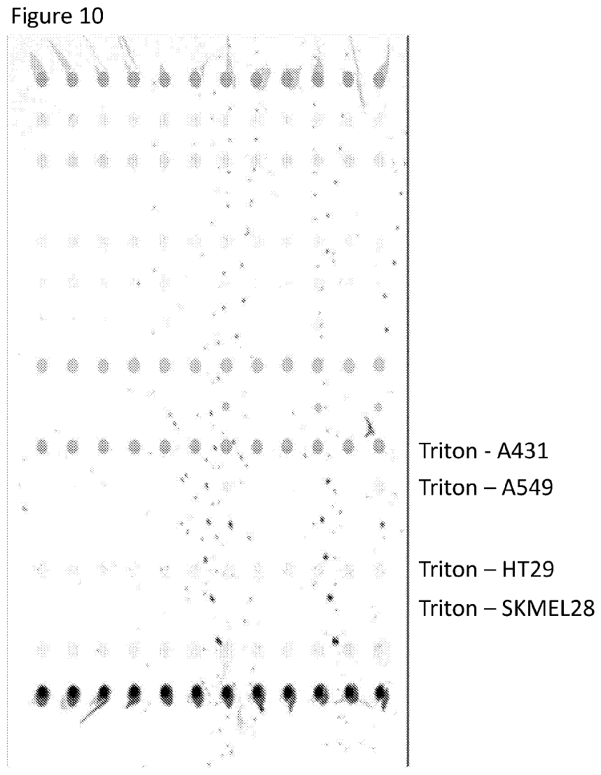

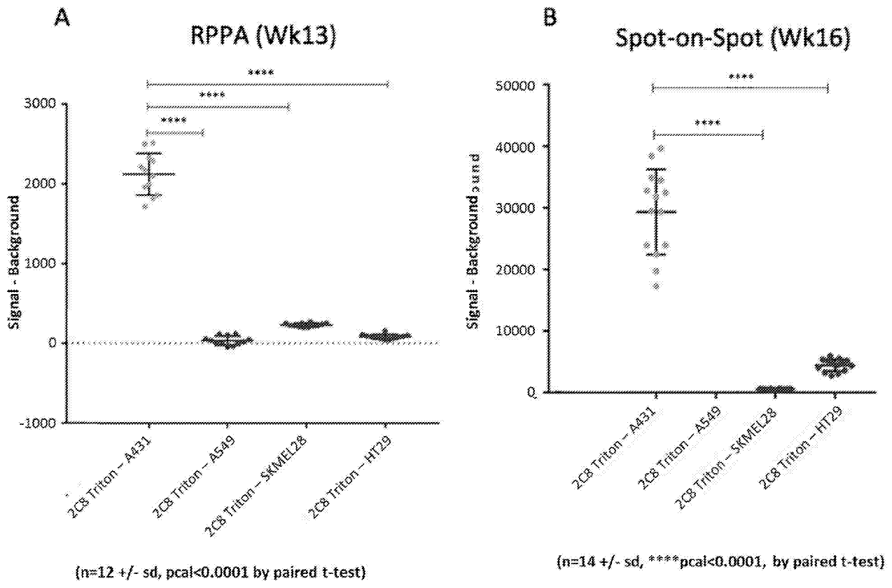

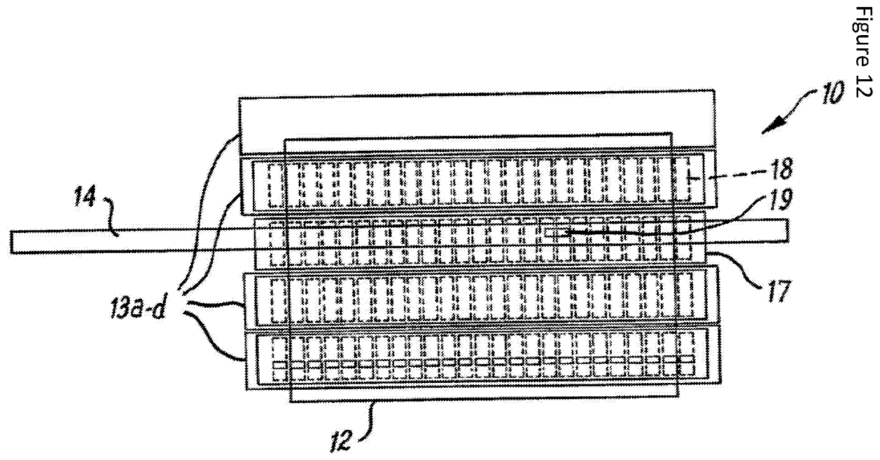

Definitions

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

All numbers expressing quantities, percentages or proportions, and other numerical values used in the specification and claims, are to be understood as being modified in all instances by the term “about.”

It should be understood that the terms “a” and “an” as used herein refer to “one or more” of the enumerated components. It will be clear to one of ordinary skill in the art that the use of the singular includes the plural unless specifically stated otherwise.

As used herein, unless the context demands otherwise, the terms “substrate”, “slide” and “plate” are to be seen as interchangeable synonyms.

### DETAILED DESCRIPTION OF THE INVENTION

A method of printing as described herein could utilise printing apparatus as discussed in patent applications WO 2004/028683 as would be known in the art. Accordingly, suitably the printing apparatus may include a platen, four cages, and a linear rail. Each cage may be a rectangular metal frame having a series of vertically stacked substrate (plate or slide) tray supports in the form of inwardly protruding ledges. The cages may be shaped to receive a number of substrate trays and each tray holding a linear array of slides to be printed. Each slide tray may be oriented lengthways across the width of the platen, and the length of the trays can be greater than the width of the platen.

In specific embodiments discussed herein, the arrangement for the slides within a tray is a linear array of, say, twenty-five slides.

The operation of the apparatus to print a first spot will now be described. The present invention provides an efficient means of printing a large number of substrates, by providing rapid means of storing, retrieving, printing and re-ordering of the slides.

The arrangement reduces the requirement for reloading the printhead with different liquids. This is particularly appropriate where the liquids are valuable and available in small quantities only. As discussed above, the loading of liquids into the printhead is inevitably wasteful in that only a proportion of each liquid is usefully printed. Inkjet printheads produce very small drops, so once liquids are introduced into the printhead, it can print a very large number of spots.

An additional advantage of this approach is that larger numbers of trays can be printed than those which fit on the platen: one or more cages can be used to store the trays when they are not being printed, to feed them onto the platen for printing and to remove them afterwards. A number of trays can be stored above each other on shelves in each cage; one or more cages can be moved vertically downwards so as to deposit the trays in turn onto the platen in preparation for printing. One or more other cages, moving vertically upwards, can remove them afterwards. The cages can perform these functions while the platen is stationary, during the printing stroke of the printhead. Thus the loading of trays onto the platen and unloading from it need not add to the time taken to print the overall number of slides in the machine; this scheme is equivalent in speed to a system using an impracticably large platen to hold all the trays. This is discussed, for example in WO 2004/028683.

A further advantage of this approach is that the cages and their motion need not be precise: if the trays are equipped with location features which engage with matching features on the platen, the act of loading each tray onto the platen ensures its accurate positioning with respect to the platen. The only parameters that need to be accurate are the printhead mounting, its motion, the platen's location features and its motion. Both of these motions are one-dimensional.

The inventors have determined that the spot on spot method described herein can provide for improved sensitivity (higher signal to noise ratio) of detection of binding between an analyte and specific binding molecule. Additionally, it is considered the spot on spot method may provide for improved discrimination between positive and negative samples (hybridoma supernatants), improved throughput and lower background.

It is considered the method can have advantages over alternative screening techniques such as RPPA (reverse phase protein array), mass spectroscopy, western blotting, or ELISA as it allows the binding of large libraries of unknown antibodies to large libraries in unknown analytes/antigens to be specifically detected in a high-throughput manner. By way of reference, it is considered that a single operator would be limited to generating an estimated 2000 data points (i.e. a specific test of the binding ability of one antigen binding agent to one analyte) in a working week using ELISA or Western Blotting which would allow the same sensitivity as the present test, whereas the present method may provide, for example 2 million data points in the same period.

It is further considered that the spot on spot method described herein can advantageously provide for a reduction in the quantity of specific binding molecule—for example antibody utilised, for example four orders of magnitude less antibody than RPPA for an equivalent test (e.g. RPPA would use ˜100 μl primary antibody to interrogate 100 lysates, whereas the present method may use ˜0.01 μl). It is considered the present method only requires 100 pL of ˜1 mg/ml of lysate and 100 pL of antibody per test. It is considered that significantly increased volumes of lysate and/or antibody would be required to test for binding using ELISA, western blot or mass spectrometry.

It is further considered the present method is advantageous as it allows a library of lysates to be screened against a library of antibodies in a single experiment, for example as different antibodies can be printed at spot positions of cell lysate, whereas RPPA would typically only allow a single antibody to be screened against a library of lysates. For example, the method of the present invention may allow 250 lysates to be screened against 250 hybridomas on a single portion of substrate (slide) containing 62,500 features, whereas RPPA would require 250 slides.

The present method can advantageously allow identification of a positive interaction between a specific binding member and an analyte. Whilst mass spectroscopy could provide increased granularity of what protein or proteins may be present in a sample, resolving these is an inherent challenge with mass spectroscopy, because the most abundant proteins (e.g., actin) will compete for detection with less abundant (but more interesting) proteins such as cytokines. Mass spectroscopy is also resource-intensive from an informatics standpoint. As noted above the method of the present invention may allow a library of lysates to be screened against a multitude of antibodies in a single experiment. Mass spectroscopy typically only allows a single antibody to be screened against a single analyte.

### EXAMPLES

### Example 1

A lysate library was normalised to 2.5 mg/mL before diluting 1:1 in 2× Protein Printing Buffer C (Arrayjet Ltd, UK) (PPBC×2). Negative control samples (BSA in PBS) were normalised to 1 mg/mL before diluting 1:1 in PPBC×2. Positive control samples (IgG in PBS) were prepared at 2 μg/mL before diluting 1:1 in PPBC×2.

200 pL of lysate library and control samples were printed onto PATH nitrocellulose slides (Grace Bio, Inc., Oregon, USA) with an Arrayjet Marathon series inkjet bioprinter (Arrayjet Ltd, UK) at 4° C. and 80% relative humidity (% RH). The slides were incubated overnight at 4° C. and 80%RH. The slides were then incubated at 30° C. for one hour. The slides were then blocked with 5% BSA in PBS-T (IgG-free) for 30 mins. Excess blocking reagent was washed off with three sequential 30 minute washes of PBS-T, PBS and distilled water. The slides were dried. 200 pL of specific binding agents (antibodies) in printing buffer were printed onto each of the lysate library ensuring that the specific binding agents are printed directly on top of the printed lysate spots. Printing conditions of 4° C. and 80% RH were used.

Excess specific binding agent was washed off with three sequential 30 minute washes of PBS-T, PBS and distilled water. The slides were incubated with labelled secondary antibody diluted 1/1000 in BSA (1% in PBS-T) (protected from light for 90 minutes) at room temperature. Excess secondary antibody was washed off with nine sequential 5 minute washes of PBS-T (×3), PBS (×3) and distilled water (×3). The slides were dried. Using a confocal laser microarray scanner (Innoscan 710 AL, Innopsys, France) the slides were scanned and data was extracted for analysis. Specific binding between the specific binding agent to the analyte is indicated by elevated fluorescence levels when compared to the negative control samples.

### Example 2

When testing the same set of lysates, the spot-on-spot method was more sensitive than RPPA (FIG. 11), indicating a higher true positive rate for the spot-on-spot technique. This was achieved by increasing the signal and reducing the background, giving an improved signal to noise ratio by approximately ten-fold. One of the key steps to achieving this is by printing the antigen binding member onto the lysate; this means that the antigen binding member is only bound to the analyte and does not bind to the entire surface of the slide, which is the case in the RPPA method.

Subsequently, when the labelled secondary antibody is applied it is less likely to create background signal.

### Example 3

An example assay was provided comprising approximately 10,000 hybridoma samples against 100 different lysate. Suitably assaying of about 1,000,000 tests a week is discussed.

Each test is carried out in duplicate. i.e. a total of up to 2,000,000 tests a week

To allow for lysate printing, five print runs of one hundred slides were utilised. This therefore provides for printing of 500 slides consisting of 5 identical slides for each lysate, i.e. 5 slides×100 lysates=500 slides.

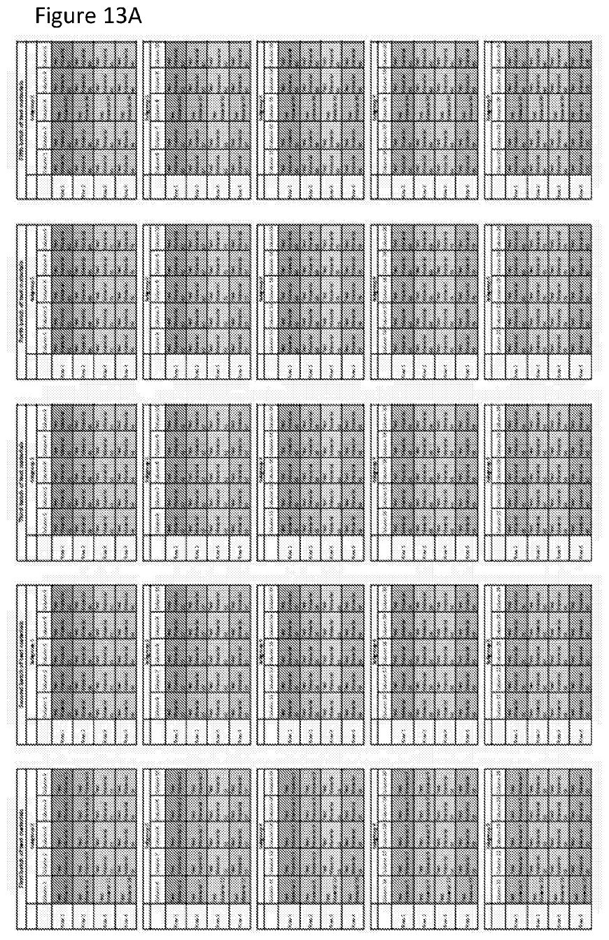

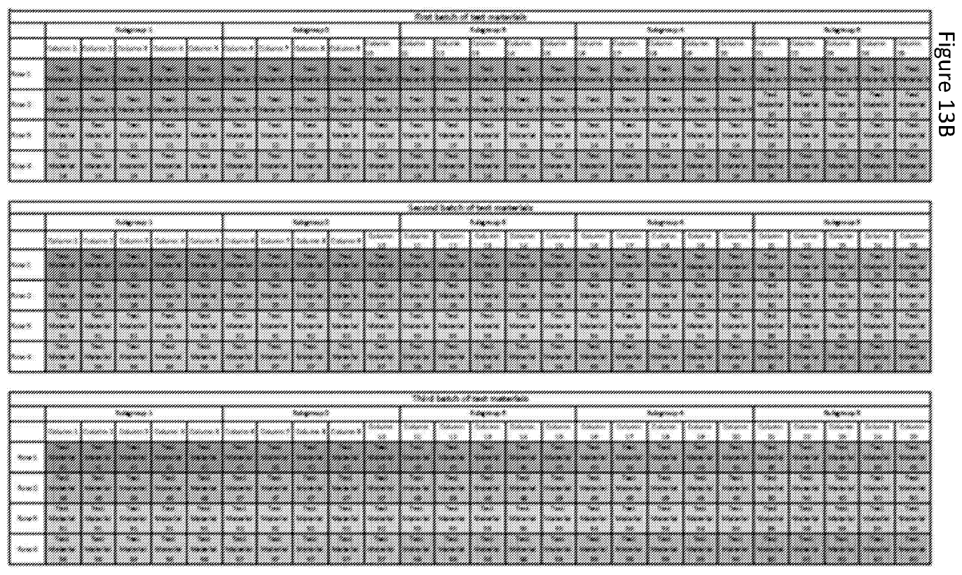

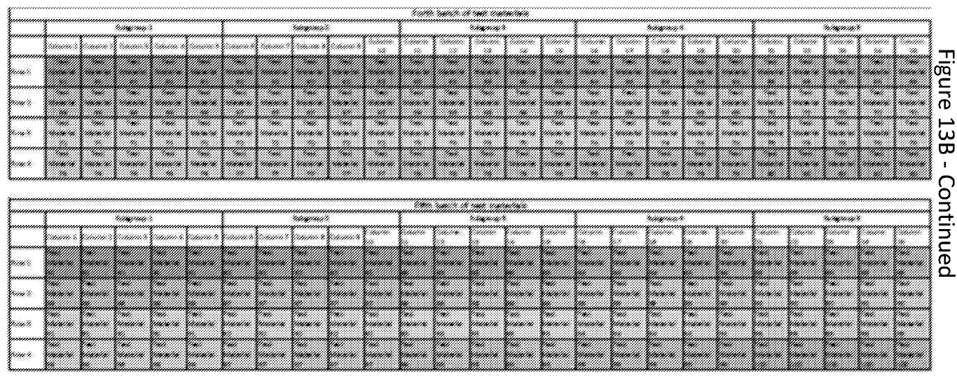

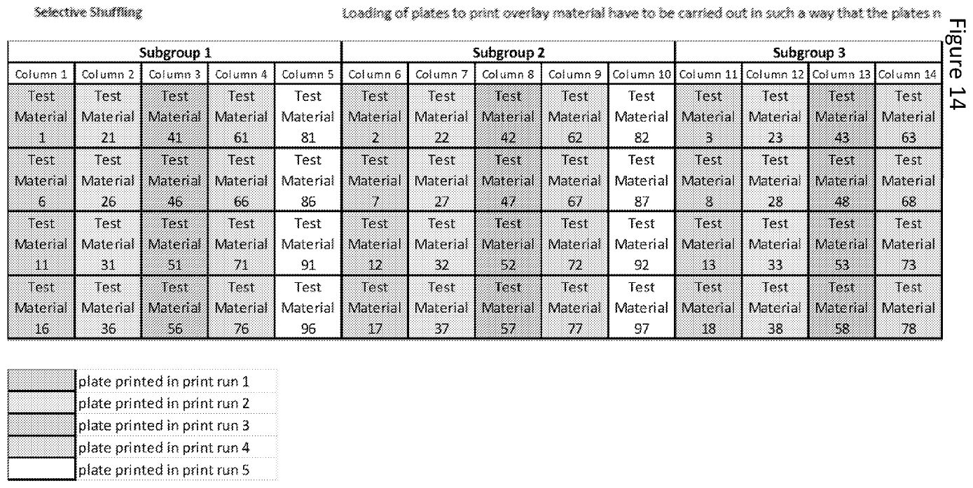

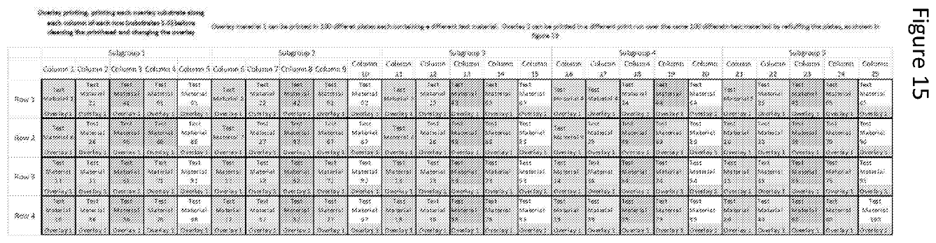

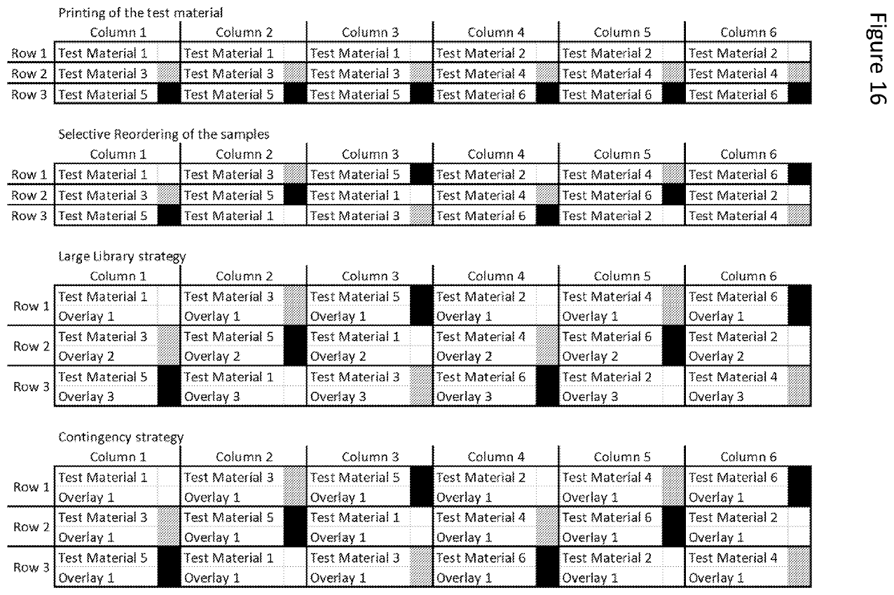

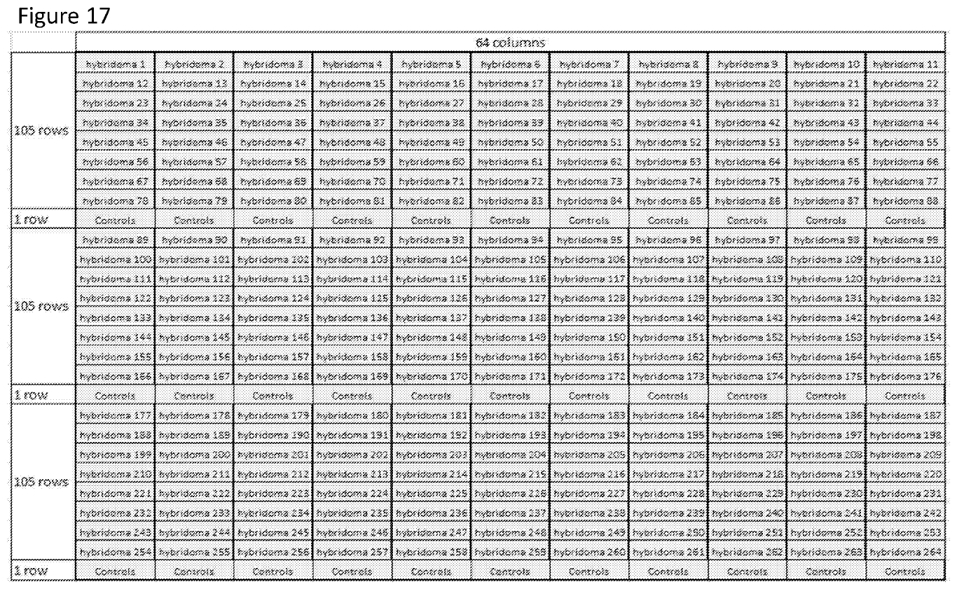

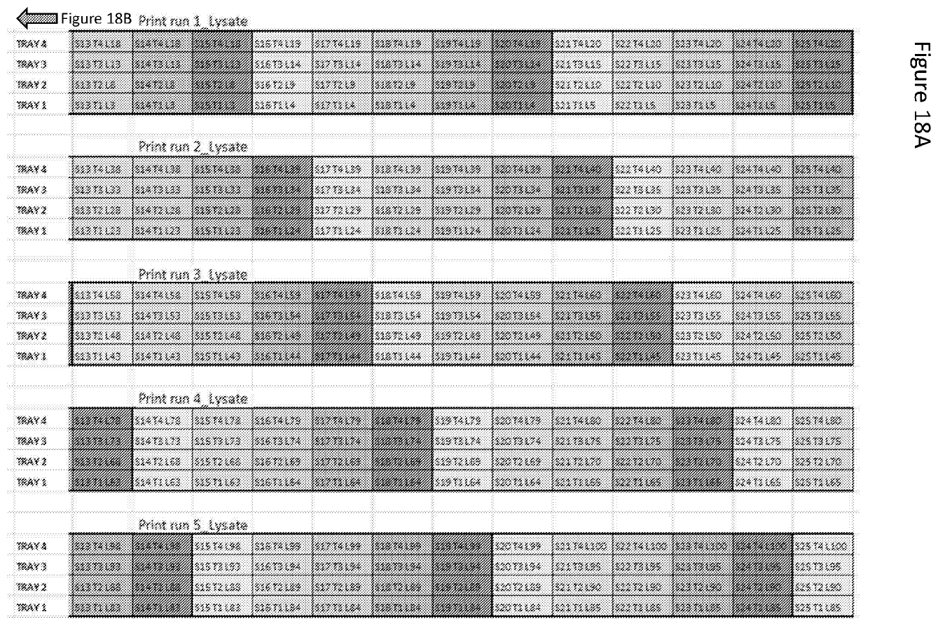

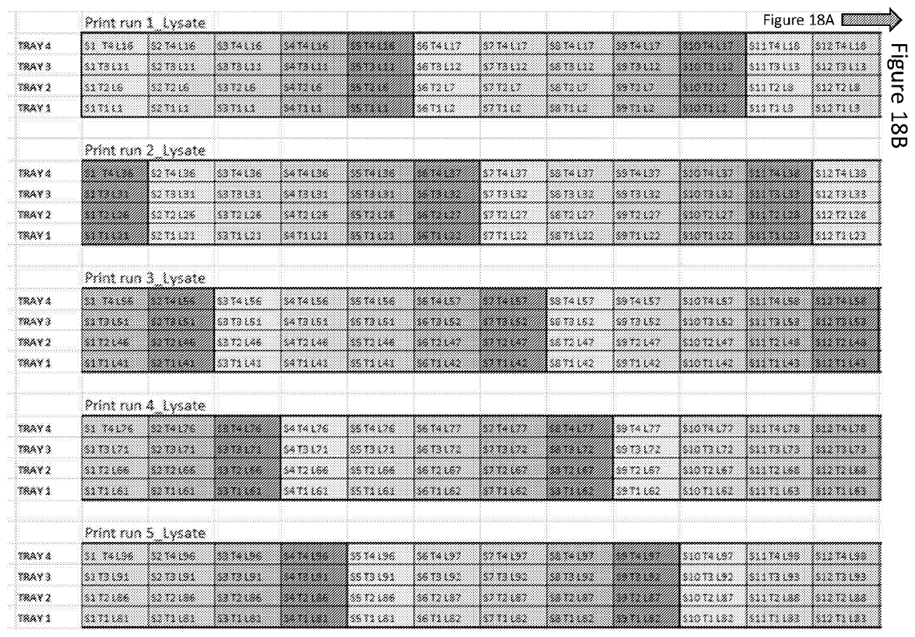

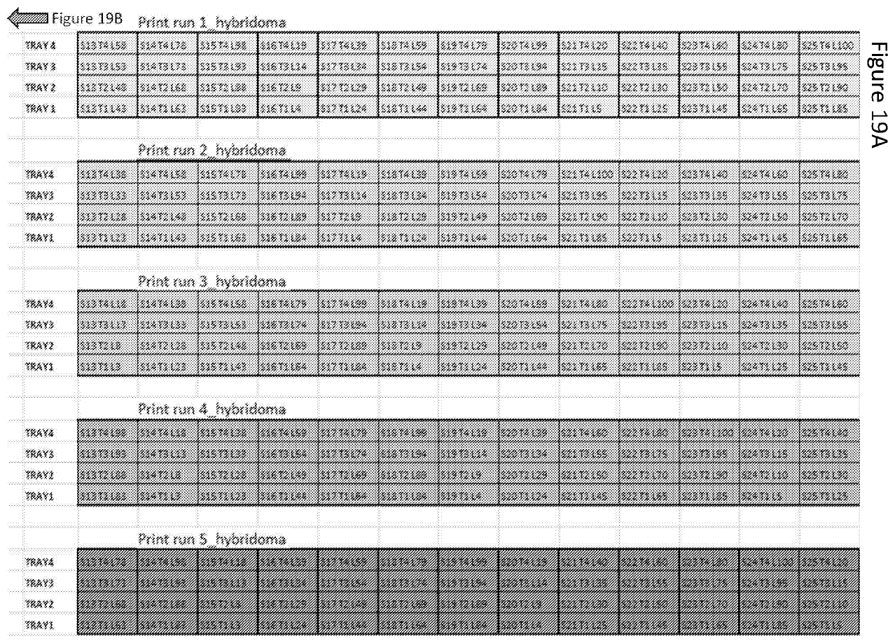

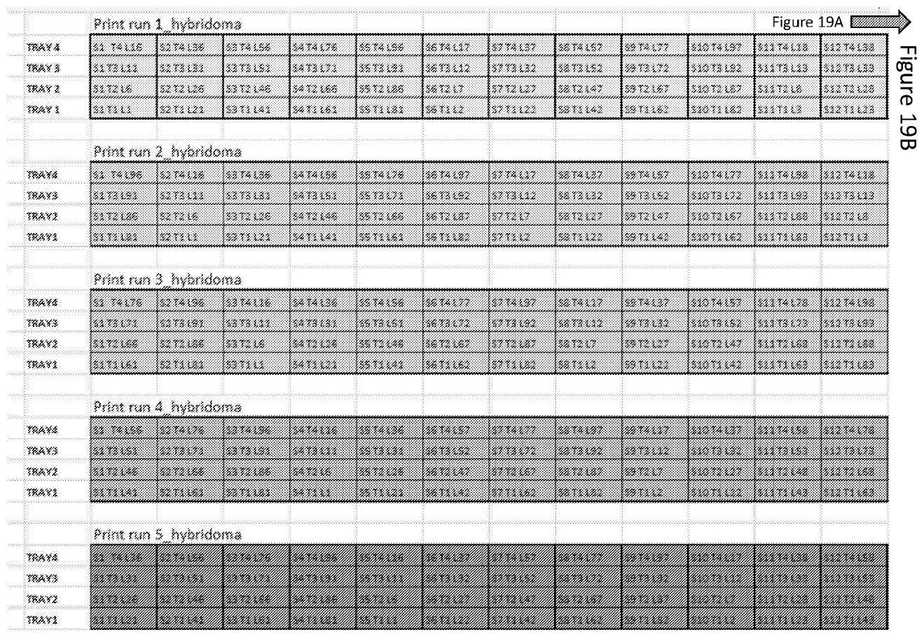

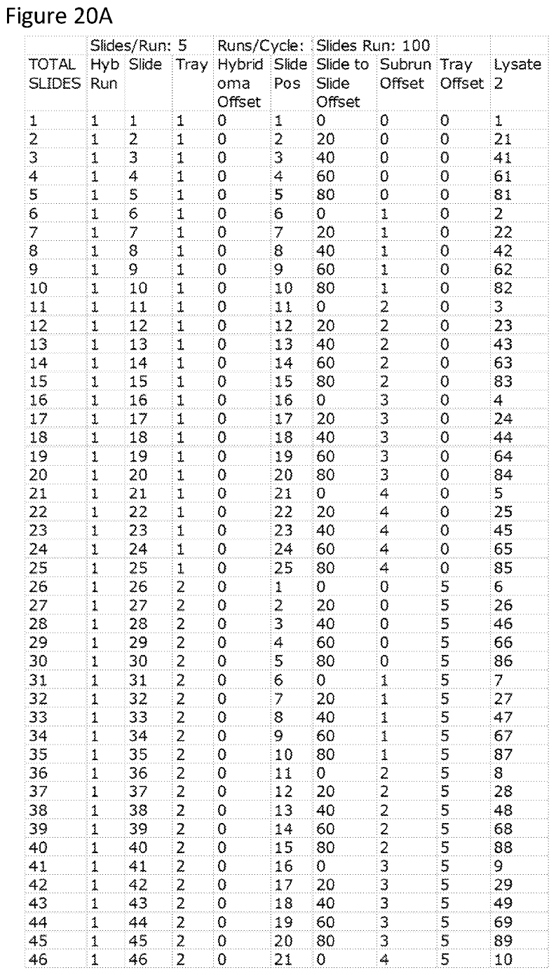

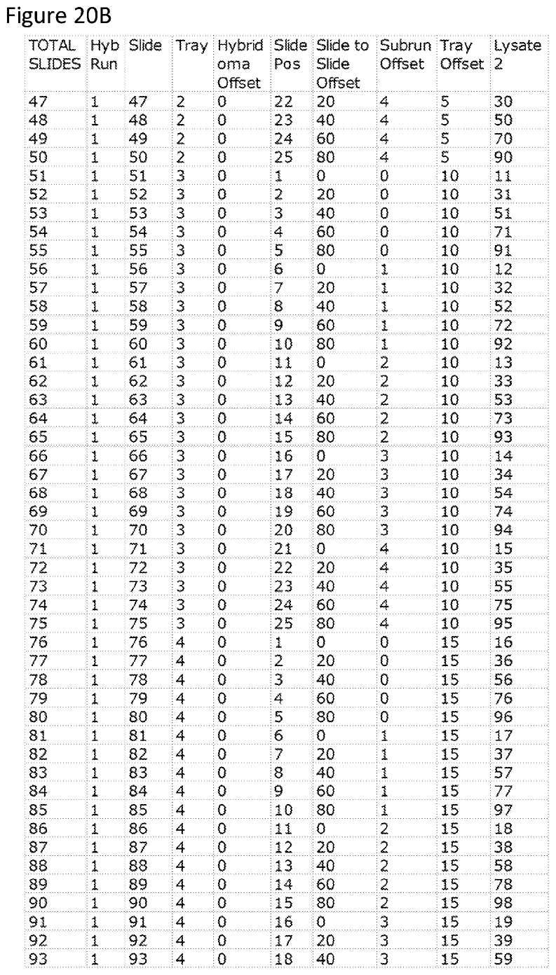

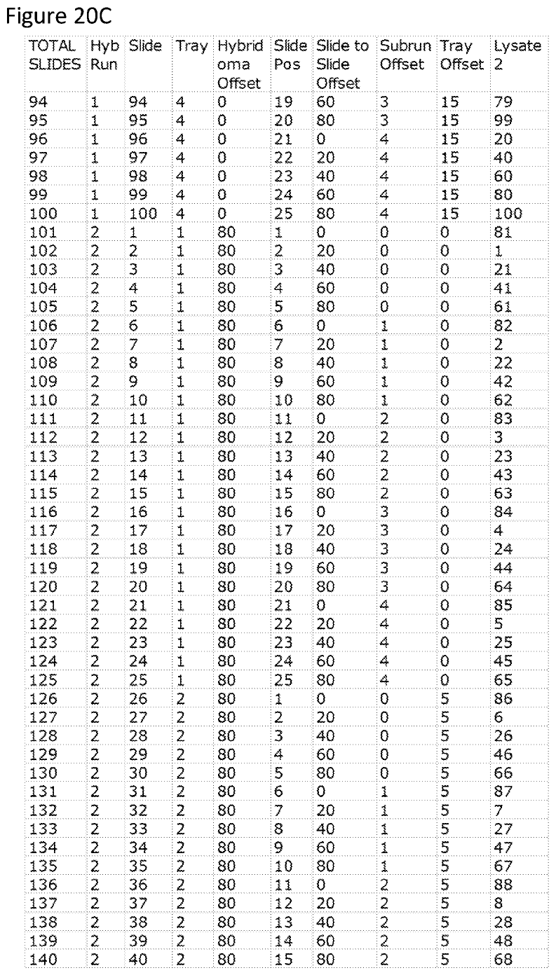

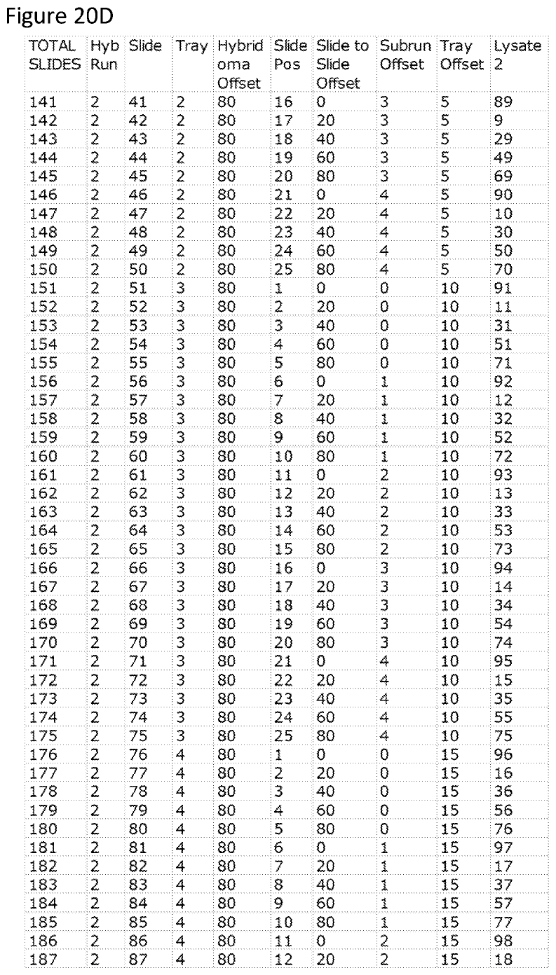

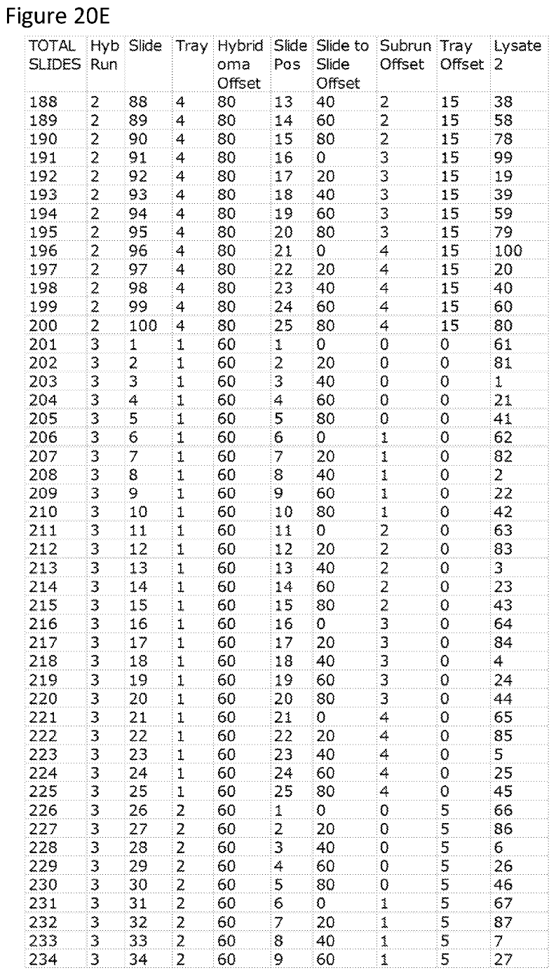

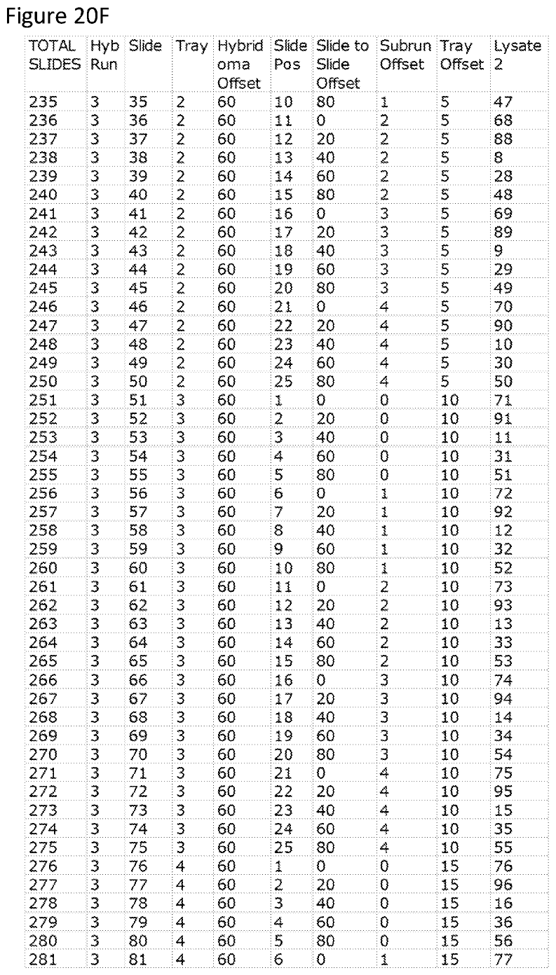

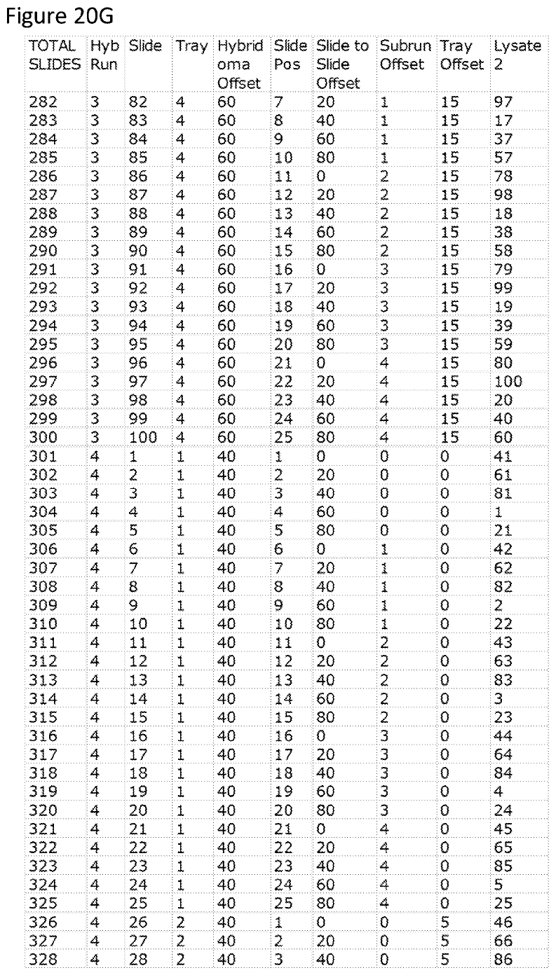

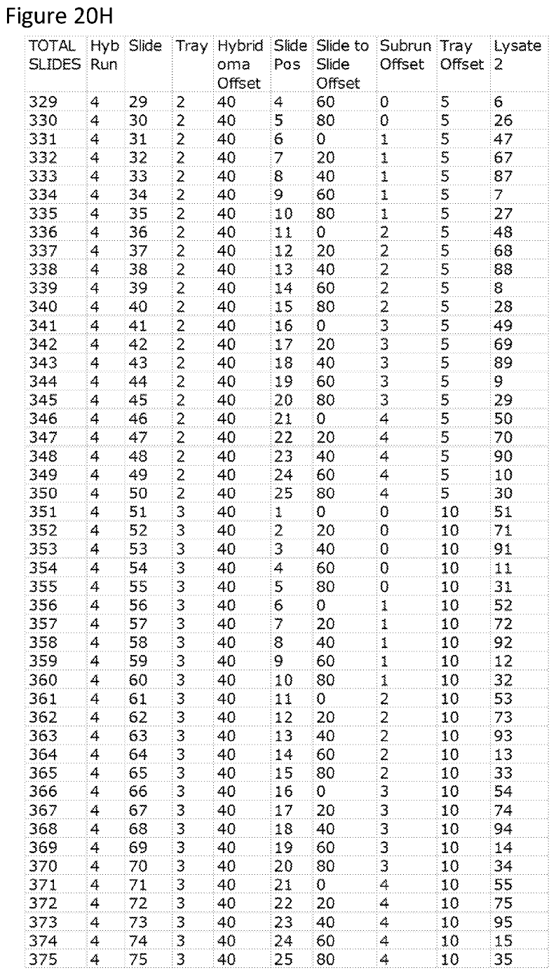

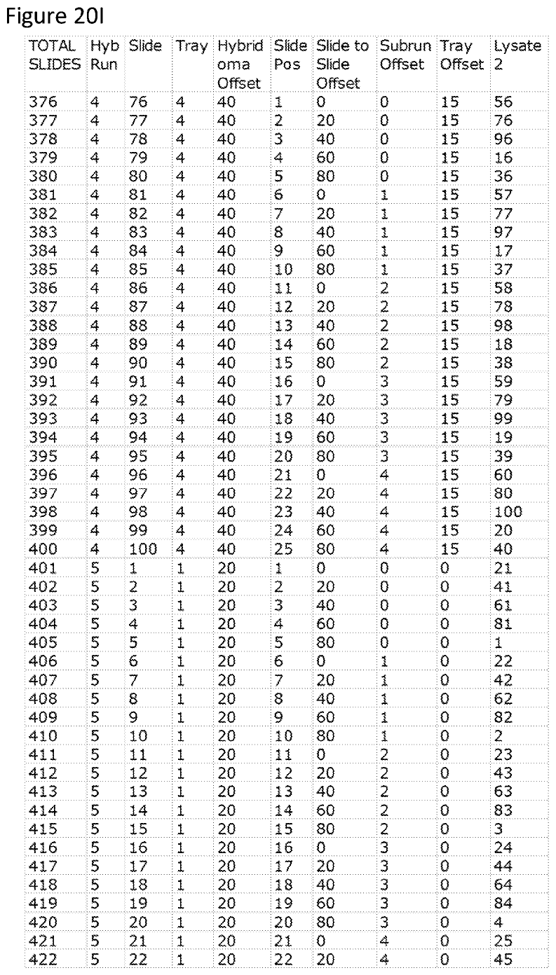

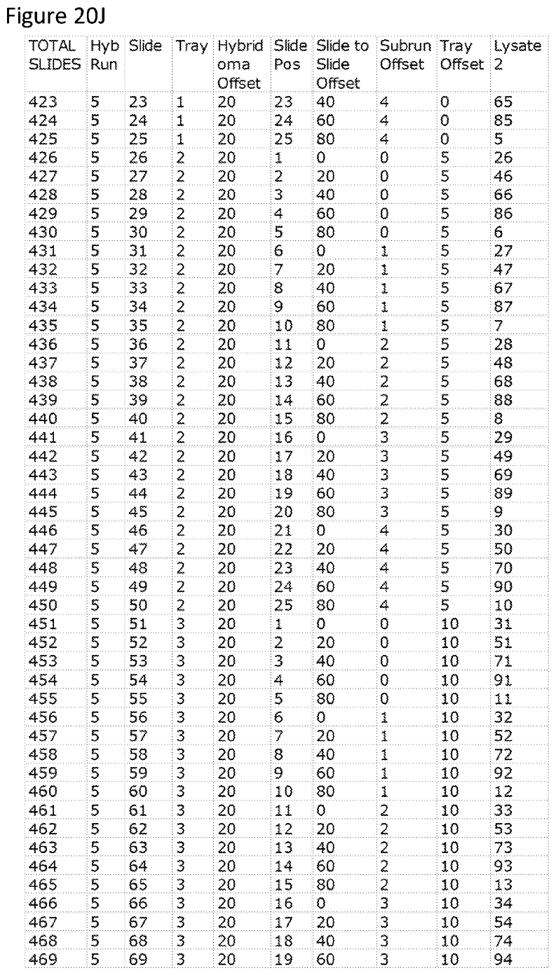

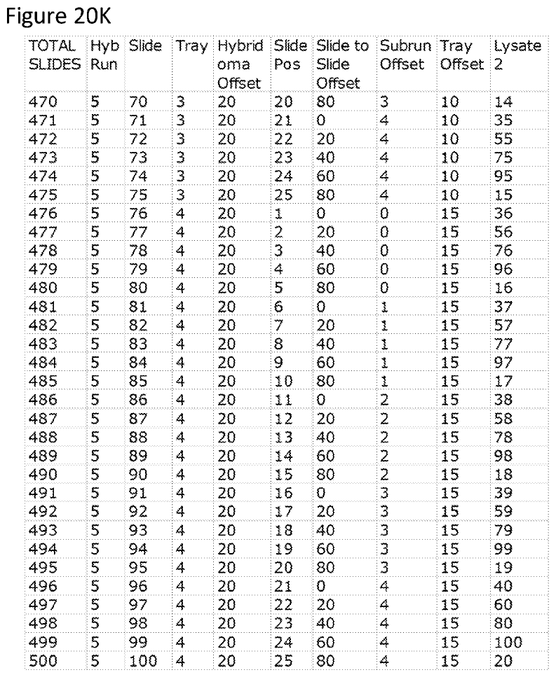

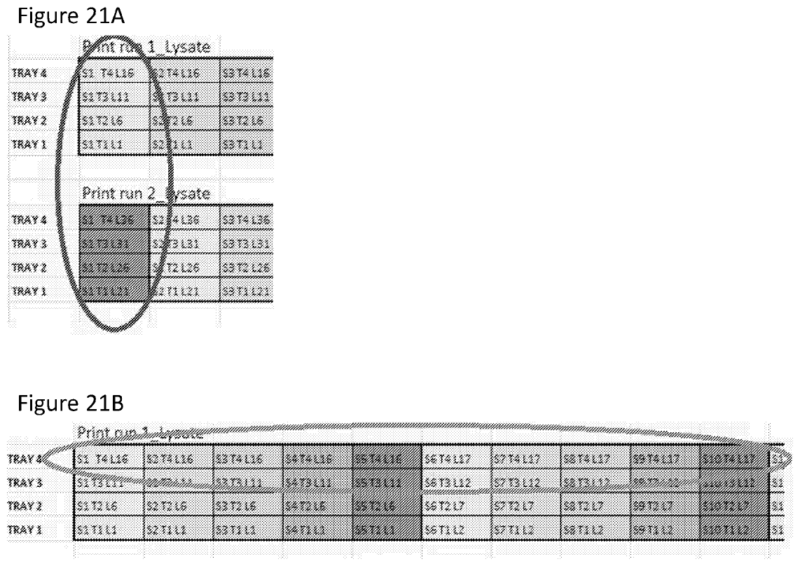

As illustrated in FIG. 13, this means that, Print run 1 will print lysate 1 to lysate 20; Print run 2 will print lysate 21 to lysate 40; Print run 3 will print lysate 41 to lysate 60; Print run 4 will print lysate 61 to lysate 80; Print run 5 will print lysate 81 to lysate 100.

The total number of spots per lysate will be 20160. This is sufficient to print 10,000 hybridomas (second test material) in duplicate, there is space to add buffer spots if required.

The second test material to be printed (in this example hybridomas) are printed with different lysates such that up to 10,080 hybridomas in duplicate are provided. Suitably the hybridomas may be provided in 10% glycerol.

Suitably an algorithm may be provided to allow for slide loading for hybridoma printing. Suitably, the system provides for a left to right shift of lysate number such that the slide position and tray are always constant. In the embodiment described herein a rotation to the right of 5 slides creates a sequence such that the slides are in the correct position for the hybridoma print run. This is illustrated for example in FIGS. 15 and 16. A colour sequence is provided to more clearly illustrate the sequence such that the hybridoma printing of the slide is provided when the slide is in the same position as lysate printing.

### Example 4

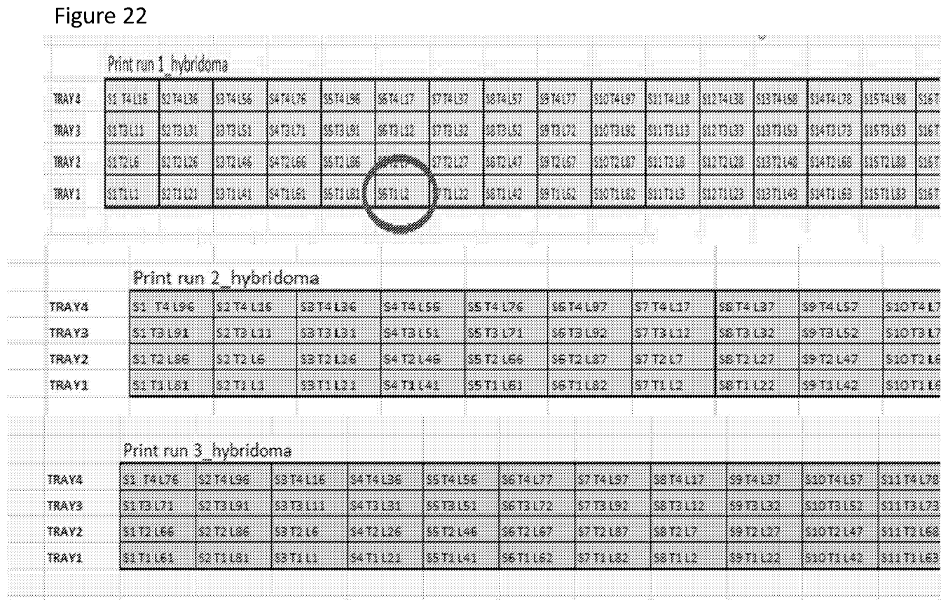

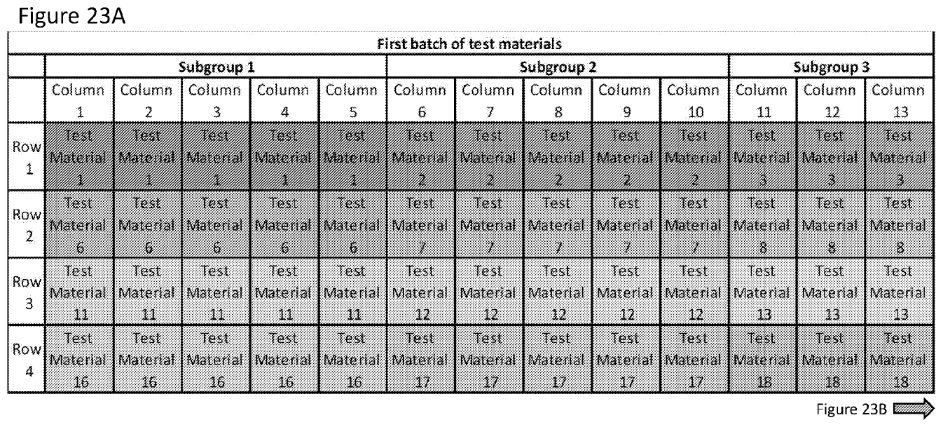

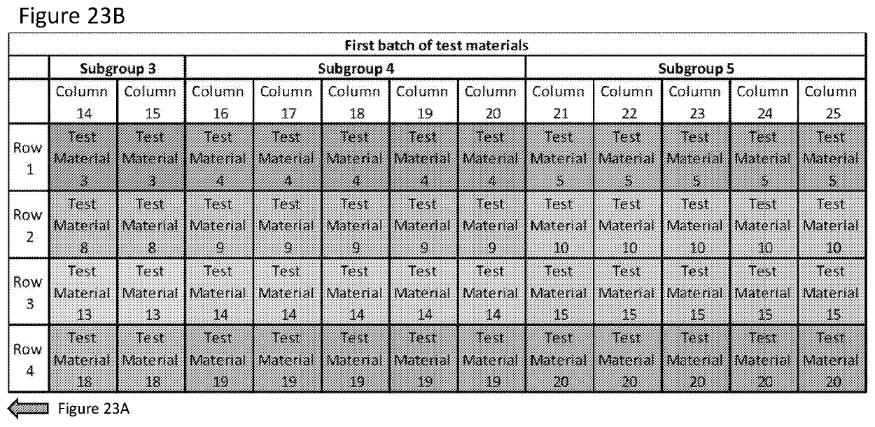

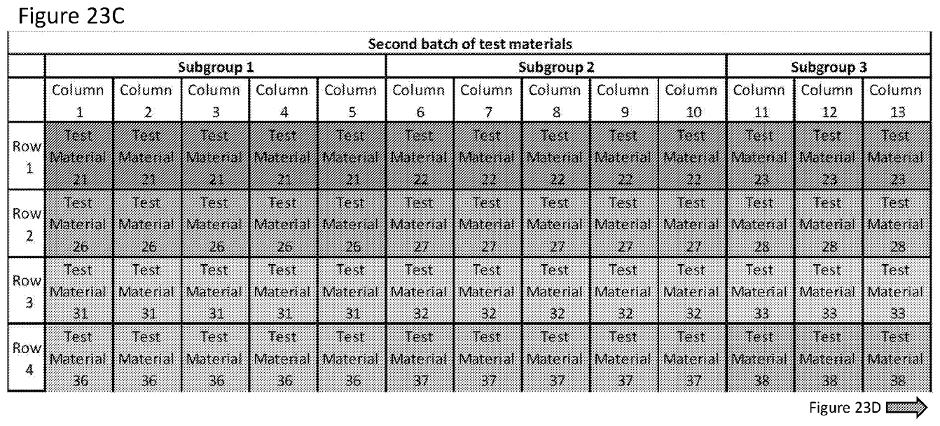

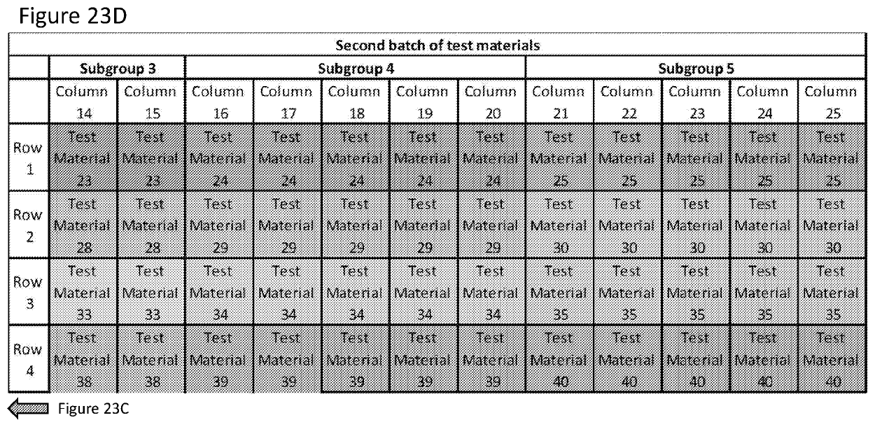

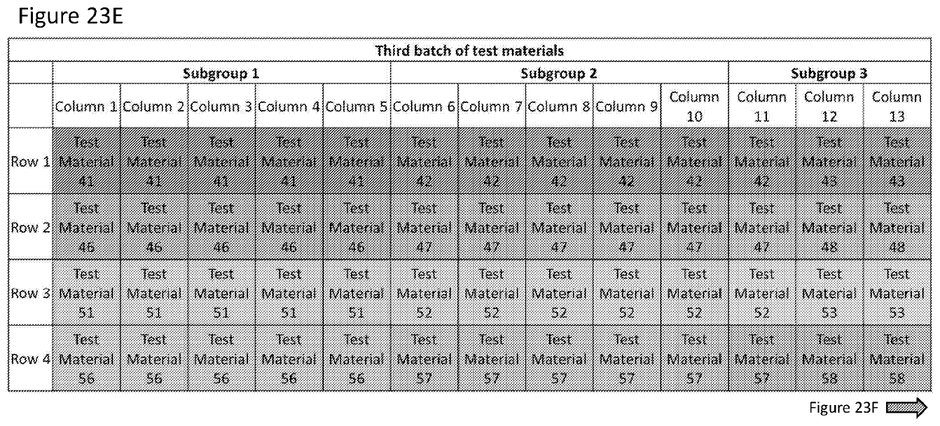

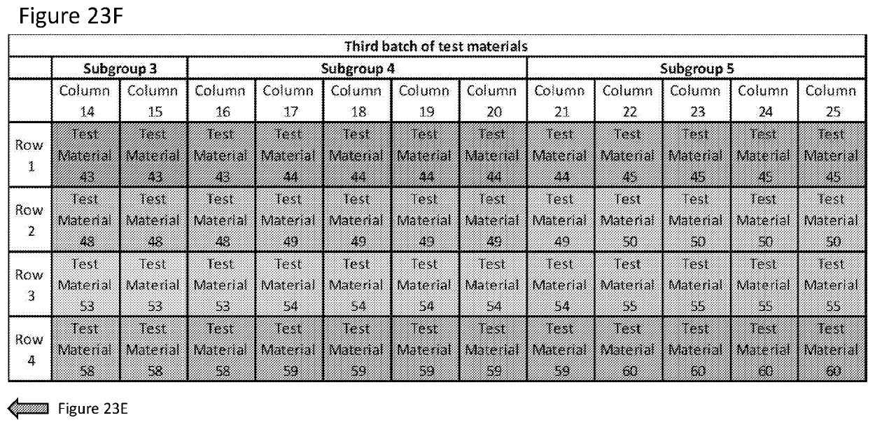

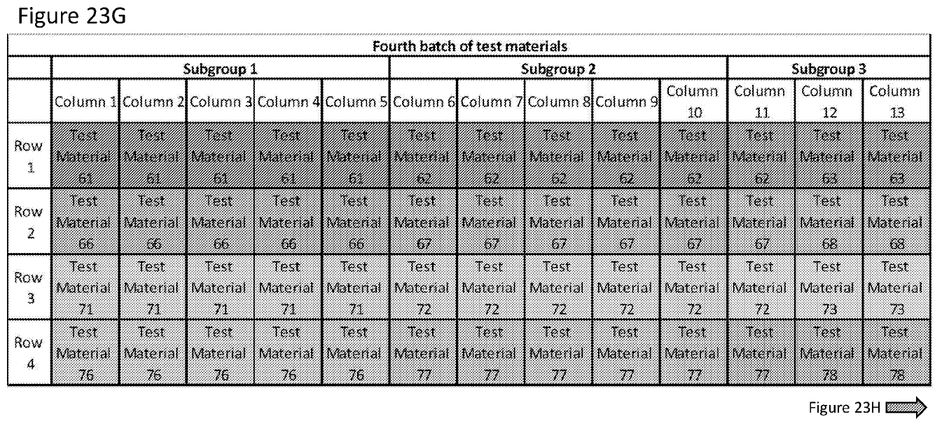

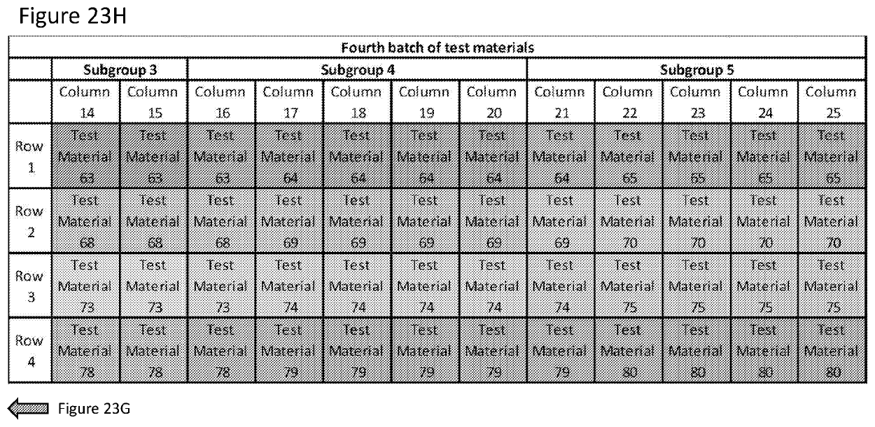

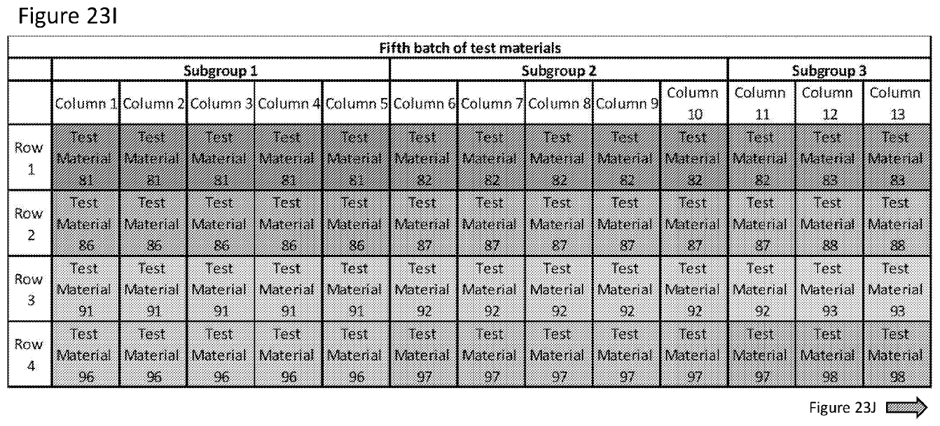

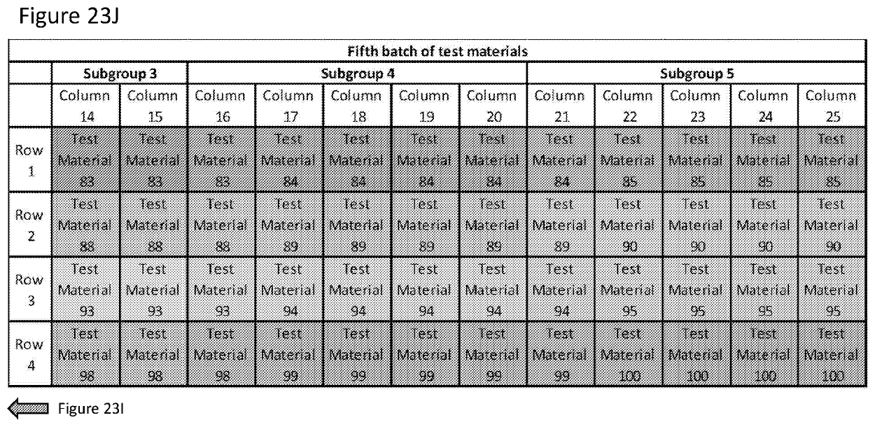

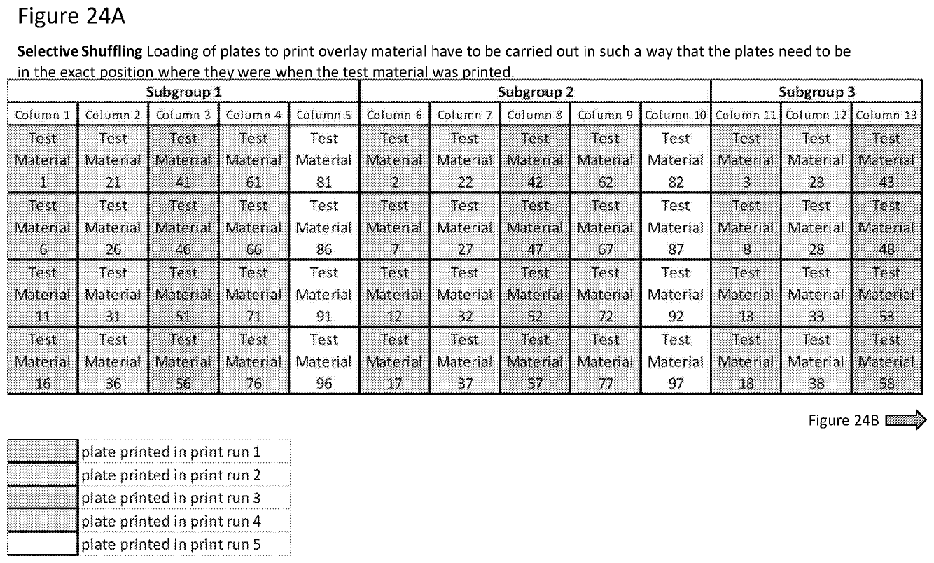

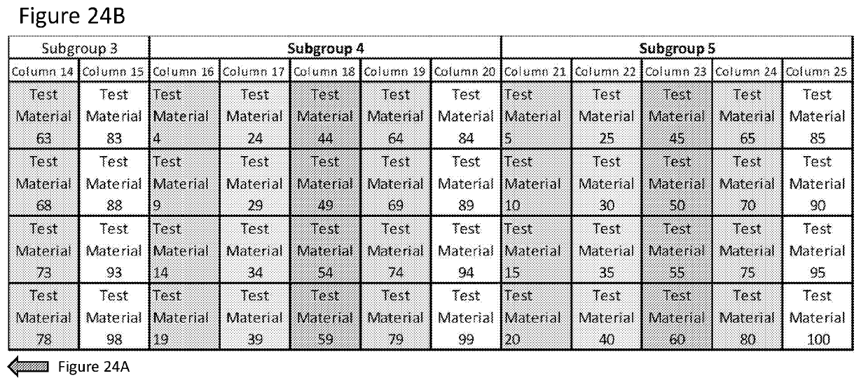

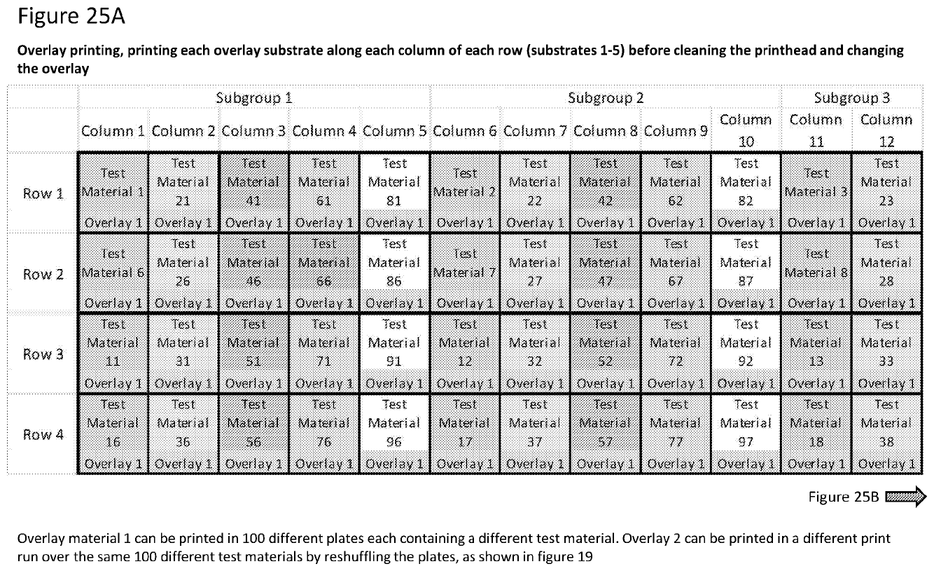

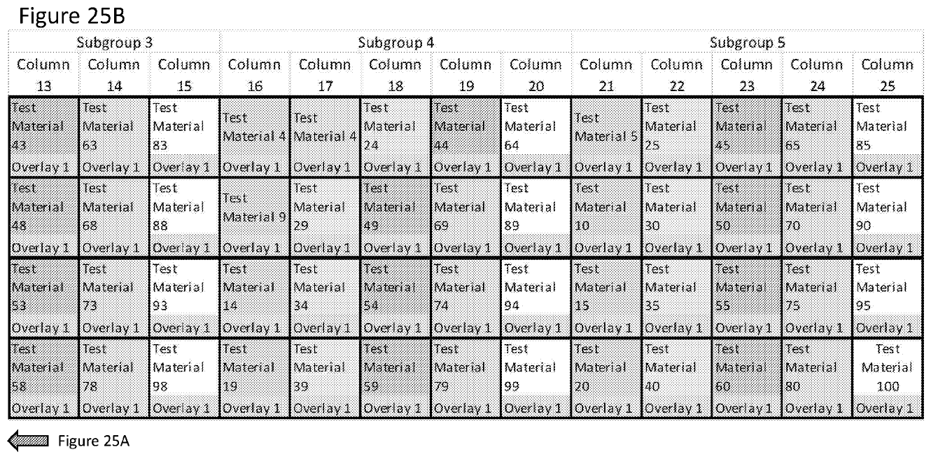

With reference to FIGS. 21 and 22, a first print run printed Lysates (L) in batches of 5, so the first five slides are all Lysate 1 L1, the slides 5 to 10 print Lysate L2, and so forth until 20 lysates are printed.

Hybridomas are then provided in overlay printing after the slides have been reordered such that the parameters S and T, are kept constant: so S1 T1 should be placed in slide 1 tray 1, etc. In this embodiment 100 different lysates are provided in one print run then were placed in same location for overlay printing where they were printed during the lysate print run, for example the first lysate 2 was printed at position 6 and thus requires to be provided in S6 T1 in the overlay printing.

Lysate 3 was printed in position 11, etc.

Because in this embodiment 20 lysates are printed in a first batch of concatenated print runs: ‘S’ and ‘T’ kept constant and the equation for ‘L’ is that the next lysate to lysate 1 should be 1+20

Within tray: in groups of 5:

Lx; L(x+20), L(x+40), L(x+60), L(x+80).

Inter-tray: The lysate should be as in the previous tray+5

Tray 1: L(x);

Tray 2: L(x+5)

Tray 3: L(x+10)

Tray 4: L (x+15)

For the following hybridomas run, a rotation between the 5 slides takes place: Lysate 1 is now in position 2, so the rotation system brings lysate 81 to position 1 (see FIG. 22 second print run)

Inter-tray is “+5” as before

For the next hybridoma run, the rotation continues and lysate 1 is now in position 3 (see FIG. 22 third print run).

As will be appreciated, a fourth print run would position lysate 1 at position 4 and the rotation system would bring

It will be evident to the skilled reader that various changes could be made to the above-described embodiments within the scope of the invention.

Various modifications and improvements can be made within the scope of the invention herein intended.

1. A method for enhancing efficiency of overlay printing of spot positions on multiple slides arranged in an array comprising the steps:
printing at least a one spot of a first test material comprising a first type of first potential binding partner pair onto a first row (r1) of slides in an array of n columns in a printing order to provide the first test material on at least slide r1n1 and a replicate slide r1n2
printing at least one spot of a second test material comprising a second type of first potential binding partner pair onto a second row (r2) of slides in an array of n columns in a printing order to provide a second test material on at least slide r2n1 and a replicate slide r2n2,
reordering the slides,
printing spots of at least a first overlay material comprising a first type of second potential binding partner pair to overlay the spots of the at least first test material and/or the spots of the at least second test material wherein when the overlay material is printed, a slide is provided at the same position in the array at which the test material was applied and the overlay material is provided without requiring movement of a printhead between rows when overlaying the first overlay material.

2. The method of claim 1 wherein when at least two different overlay materials are printed the slide is provided at the same position in the array at which the first test and material was applied.

3. The method of claim 1, wherein the test material comprises an analyte, in particular a cell, cell-derived product, cell lysate, a protein, a protein fragment, a receptor provided on an intact cell, a receptor provided in a cell lysate, a fusion protein, a nucleic acid sequence or the like.

4. The method of claim 1, wherein a second potential binding partner (provided in the overlay material) is a specific binding molecule selected from a group comprising an antibody or a fragment thereof, a small molecule, an aptamer, or a nucleic acid molecule.

5. The method of claim 1, wherein a subgroup of a number of slides (Sr)′ may be printed with a first test material (t1) to provide replicate slides with each replicate slide providing a first position of a column in a row (for example: r1n(Sr)′t1, r1n(Sr)′t1, r1n(Sr)′t1, r1n(Sr)′t1, r1n(Sr)′t1).

6. A method of claim 5 wherein the subgroup is 5 slides, such that in the method, in each row, five replicate slides are printed with a first test material (t1) with each replicate plate providing a first position of a column in a row: r1n1t1, r1n2t1, r1n3t1, r1n4t1, r1n5t1.

7. The method of claim 6 wherein the overlay material is printed following reordering of the slides determined based on the following input
Binding partner Print Run (overlay Hybridoma Print Run Number (H))
Slide Number (S)
Tray Number (T)
Slides per subrun (Sr)=5
Runs/cycle (Rc)=20
Slides per total run (St)=100
Slides per tray=25
Binding partner Offsset (e.g. HybridomaOffsset)=MOD(Rc*(Sr−(H−1)), St);
Which provides (based on above):
Binding Partner Offsset (e.g. HybridomaOffsset)=Ho=MOD(20*(5−(H−1)), 100);
SlidePosition=Sp=MOD(S−1, 25)+1;
SlideOffset=So=MOD((SlidePosition−1)*20, 100);
SubrunOffset=Sro=QUOTIENT(Sp'11,5);
TrayOffset=To=(T−1)*5;
Test Material (e.g. Lysate)=L=MOD(Ho+So+Sro+To, 100);
where MOD is the module operation (remainder after division of one number by another).

8. The method of claim 1, for detection of analyte to a specific binding molecule comprising:
incubating the printed test material and overlay material to allow binding between an analyte and specific binding member provided in the test material and overlay material to occur,
detecting any binding between an analyte and specific binding member provided in the test material and overlay material.

9. The method of claim 8 wherein the detecting step is an immunohistochemical detecting step, optionally wherein the detecting comprises detecting fluorescence, colorimetry, quantum dots, biotin/avidin, surface plasmon resonance to identify binding.

10. The method of claim 1, wherein there is provided a blocking step prior to the printing of overlay material.

11. A method of diagnosing a disorder, the method comprising
determining the presence of an analyte in a test material sample using a method of claim 1,
wherein when binding of a specific binding molecule provided in an overlay material and analyte in the test material is detected it is indicative of the disorder.

12. A system to provide the method of claim 1, wherein the system comprises a printer adapted to print a test material onto a substrate, to provide a first row (r1) of slides in an array of n columns in a printing order to provide the first test material on at least slide r1n1 and a replicate slide r1n2, to print at least one spot of a second test material onto a second row (r2) of slides in an array of n columns in a printing order to provide a second test material on at least slide r2n1 and a replicate slide r2n2, to print spots of at least a first overlay material to overlay the spots of the at least first test material and/or the spots of the at least second test material wherein when the overlay material is printed, a slide is provided at the same position in the array at which the test material was applied and the overlay material is provided without requiring movement of a printhead between rows when overlaying the first overlay material.

13. The system of claim 11 wherein the system comprises an environmentally controlled module around the printer.
